(19) United States
(12) Patent Application Publication
(10) Pub. No.: US 2015/0267210 A1
(43) Pub. Date: Sep. 24, 2015

(54) ELECTROCOMPETENT CELLS AND PREPARATION THEREOF

(71) Applicant: GENEBRIDGES GMBH, Heidelberg (DE)

(72) Inventors: Rolf Mueller, Blieskastel (DE); Qiang Tu, Saarbruecken (DE); Youming Zhang, Saarbruecken (DE); Francis Stewart, Dresden (DE); Jun Fu, Dresden (DE)

(21) Appl. No.: 14/435,950

(22) PCT Filed: Oct. 15, 2013
(86) PCT No.: PCT/EP2013/071546

§ 371 Date: Apr. 15, 2015

Related U.S. Application Data
(60) Provisional application No. 61/714,372, filed on Oct. 16, 2012.

(51) Int. Cl. C12N 15/74; C12N 15/70; C12N 1/20

(52) U.S. Cl. CPC C12N 15/743; C12N 1/20; C12N 15/74; C12N 15/70

(57) ABSTRACT

The present invention provides a method of preparing electrocompetent cells from Gram-negative bacteria characterized in that the bacteria are prepared under room temperature. Also provided are electrocompetent Gram-negative bacteria and kits which comprise the electrocompetent bacteria.

### FIELD OF INVENTION

The present invention relates to Gram-negative bacterial cells and their preparation to take up exogenous materials. More particularly, the present invention is related to the field of electroporation and cellular materials prepared for electroporation. In another aspect, the present invention is related to the packaging and delivery of the cell materials used in the field of electroporation.

### BACKGROUND OF THE INVENTION

Competent cells are cells which are able to take up exogenous materials. Routine procedures in biotechnological laboratories involve the daily use of various competent cells for cloning, propagation and preparation of plasmid DNA, construction of genomic libraries, protein expression, and mutagenesis. Different types of competent cells are available commercially, including bacterial, insect, yeast and mammalian cell lines. The Gram-negative bacterium Escherichia coli is one of the most extensively used competent cells.

Non-competent cells can be made competent to take up exogenous materials chemically or via electroporation. For example, E. coli cells can be rendered competent by washing with divalent cations such as Ca²⁺ at 0° C. or ice-cold (Hanahan, Studies on transformation of Escherichia coli with plasmids, J Mol. Biol. 1983 166(4):557-580). In contrast, electroporation is the application of electric field pulses to cells and tissues to cause structural rearrangement of the cell membrane. The high voltage causes the cellular membrane to be transiently permeabilized, allowing the foreign material to enter the cell (see, e.g., Andreason and Evans, Biotechniques, 6: 650-660 (1988)). Electroporation is commonly used to transform cells (see, e.g., Dower et al., Nucleic Acids Research, 16: 6127-6145 (1988); Taketo, Biochimica et Biophysica Acta, 949: 318-324 (1988); Chassy and Flickinger, FEMS Microbiology Letters, 44: 173-177 (1987); and Harlander, Streptococcal Genetics, eds. Ferretti and Curtiss, American Society of Microbiology, Washington, D.C., pp. 229-233 (1987)). Although introduction of nucleic acids into cells in vitro is the most common application, electroporation has also been used to introduce other molecules, such as proteins, drugs, viruses, fluorescent dyes, carbohydrates into cells.

Gram-negative bacteria are frequently used in recombinant biotechnology. Gram-negative bacteria differ from Gram-positive bacteria in the external structures. Gram positive bacteria can be stained dark blue or violet by Gram staining, while Gram-negative bacteria cannot retain the crystal violet stain and instead take up the counterstain and appear red or pink. Approximately 40 layers of peptidoglycan are found in the cell walls of Gram-positive bacteria, accounting for approximately 50% of the total cell wall thickness. In contrast, Gram-negative bacteria generally contain maximum of two to three layers of peptidoglycan, accounting for 5-10% of the total cell wall thickness. Although Gram-negative bacteria contain an additional outer membrane, the cell wall of Gram-positive species is generally thicker and more resistant to physical stress. Due to this difference, different electroporation techniques were designed for Gram-positive bacteria to penetrate this physical barrier. Notably, electroporation of Gram-positive cells require varied conditions for different species and strains.

Protocols for electroporating Gram-negative bacteria are described in Nikoloff (ed.) Methods in Molecular Biology 47, Humana Press, Totowa N J (1995). Generally, cells are grown to a suitable density, harvested, and followed by a series of washes to remove culture medium. For storage, the cells are then suspended in a small volume of glycerol, divided into tubes, frozen and stored long-term under −70° C. or lower. Known methods of preparing electrocompetent Gram-negative bacteria require the cells to be performed at ice-cold temperatures, as well as the equipments and the wash solutions to be pre-chilled.

Competent cells produced using procedures developed to date, as well as commercially available competent cells (e.g. E. coli), are being shipped or delivered frozen at about −70 to −80° C. The conventional wisdom is that storage at a higher temperature, for example at −20° C. or higher, will result in a significant decrease in viability and transformation efficiency. Cells can lose up to 90% of transformation efficiency after only 24 hours of storage at −20° C.

This typically requires the use of solid carbon dioxide (dry ice) within the packaging in order to sustain such the low storage temperature. This suffers from the drawback of increased cost of shipping. Furthermore, storage at −70° C. or below requires expensive and space-inefficient cold freezer units which may not be available. Freezing also entails the thawing step to be performed once the cells are removed from storage.

Attempts have been made to provide competent cell which can be stored at higher temperatures, since many labs do not have access to ultra cold freezer. For example, U.S. Pat. Appl. No. 2005/0053911 discloses a method of generating storage-stable competent cells by drying competent cells in the presence of a glass-forming matrix at a temperature above freezing. U.S. Pat. No. 5,891,692 describes a method of storing competent bacterial cells at −20° C. to 4° C. without appreciably losing transformation efficiency or viability. The method relies on altering the fatty acid content of the bacteria and requires transforming bacterial cells with exogenous E. coli fabB genes. This method however requires the manipulation of the bacterial cell genome individually. Accordingly, there is still a need in the industry for alternative methods of preparing electrocompetent cells from Gram-negative bacteria, where the produced cells can be shipped at higher temperatures.

There is also a need to provide cells whose competency is improved. Efforts have been made in the past to increase transformation efficiency of the cell. Hanahan et al (Studies on transformation of Escherichia coli with plasmids. J Mol. Biol. 1983; 166(4):557-80, 1983) first examined factors that affect the efficiency of transformation, and devised a set of conditions for optimal efficiency applicable to E. coli. Several factors are known to affect transformation efficiency, including plasmid size, type of cells, growth of cells, and condition of transformation. Some researchers focus on developing new strains having higher transformation efficiency, while others devises new protocols which leads to such improvements. It is therefore desirable to provide a method of preparing electrocompetent cells from Gram-negative bacteria which exhibit increased transformation efficiency.

One object of the present invention is to provide a method of preparing Gram-negative bacterial cells for electroporation where the prepared cells do not require the use of dry ice to maintain the shipping or delivery temperature and where their competency is not compromised.

Preparation of cells for electroporation can be a tedious process. It is therefore one object to provide a novel method that is simplified, efficient, fast, cheaper, or convenient.

### SUMMARY

In a first aspect, the present invention provides a method of preparing electrocompetent cells from Gram-negative bacteria, in particular from Proteobacteria, and more particularly from Escherichia. The method comprises growing Gram-negative bacterial cells in a medium, isolating the cells, and washing the cells under about 20° C. to about 30° C.

The present invention is based on the finding that preparing electrocompetent Gram-negative bacteria this way has multiple surprising advantages. It has always been understood that for maximum efficiency of transformation, it is absolutely crucial that the temperature of the bacteria must not rise above 4° C. and preferably not above 0° C. at any stage. It is therefore expected that if this temperature is exceeded there would be detrimental effect on cell viability as well as on transformation efficiency. Astonishingly, the inventors have discovered, contrary to all of the teachings of prior art, that preparing E. coli under elevated temperature not only eliminates the need for dry ice for shipping, but also leads to the increase transformation efficiency. This finding is furthermore confirmed in other Gram-negative bacteria cells. Accordingly, the inventors have discovered a novel method of preparing electrocompetent cells which is widely applicable to all Gram-negative bacteria.

Preparation under room temperature has been tried only in Gram-positive bacteria including Mycobacterium marinum (Talaat et al, Transformation and transposition of the genome of Mycobacterium marinum, Am. J. Vet. Res. 2000; 61(2):125-8) and Clostridium perfringens (Jiraskova et al., Rapid protocol for electroporation of Clostridium perfringens, J. Microbiol. Methods. 2005; 62(1):125-7). However, for Gram-positive bacteria optimal electroporation temperatures often vary depending on the species or strains. For example, for mycobacteria, electroporation at 37° C. was found to be optimal for M tuberculosis, whereas electroporation at 0° C. is optimal for fast growing mycobacteria such as Mycobacterium smegmatis (Talaat et al.) On the other hand, for Mycobacterium marinum, electroporation at room temperature was found to increase in transposition efficiency with the addition of ethionamide (Talaat et al). Likewise, for clostridial bacteria, the conditions required for electroporation vary depending on the species and strains. For Clostridial perifrigens, it was found that harvesting cells early in in the logarithmic stage of growth, keeping the cells at room temperature and absence of post-shock incubation on ice increased transformation efficiency. In any case, whether electroporation under room temperature would be useful for a given Gram positive bacteria species must be tested individually.

Preparation under room temperature has never been suggested nor demonstrated successful for Gram-negative bacteria.

The present invention is drawn to a method of preparing electrocompetent cell from Gram-negative bacteria characterized in that the preparation is taken under room temperature. In another aspect, the present invention is drawn to a method of improving the competence of an electrocompetent Gram-negative bacterial cell comprising preparing the bacteria under room temperature. As defined herein, the term competence refers to the ability to take up exogenous material by electroporation. This can be represented by transformation efficiency as described in the present invention.

The washed bacteria according to the present invention can be immediately subjected to electroporation or to preparation for distribution or storage. In one preferred embodiment, the method further comprises drying the washed Gram-negative bacteria. Drying bacterial cells and storing them in seals containers prevent ingress of water and allow storage. Heckly (Preservation of bacteria by lyophilization. Adv Appl Microbiol. 1961; 3:1-76) describes the application of freeze-drying and liquid-drying to the preservation of bacteria. Many other approaches of drying are known in the art. Rehydration of the bacteria is carried out prior to electroporation.

Gram-negative bacteria prepared by the present invention can be stored in a wide range of temperatures. In a further preferred embodiment, the present method further comprises storing the Gram-negative bacteria under about −80° C. to about 30° C., and preferably under about −20° C. to about 25° C., more preferably about 0° C. to about 4° C., and most preferably at about 4° C. It is not necessary to store the bacteria under −70° C. immediately after washing. Storing at −70° C. or less imposes significant burden on distribution of the bacteria, its subsequent storage and use. In the present invention, the electrocompetent cells can be delivered under about 0-4° C. (for example with wet ice). In a particularly preferred embodiment the method comprises both drying the Gram-negative bacteria and storing the Gram-negative bacteria.

The method described herein is useful for preparing electrocompetent cells for electroporation. Thus, in a second aspect, the invention also provides an electrocompetent cell prepared by growing the Gram-negative bacterial cells in a medium, isolating the cells, and washing the cells under about 20° C. to about 30° C. Optionally the preparation further includes drying the cell.

In another aspect, the present invention is related to electrocompetent cells having an absolute transformation efficiency of at least 7×10⁻⁶. Such electrocompetent cells can be obtained by using the methods described in the present invention. In one embodiment, the cells have an absolute transformation efficiency of at least 1×10⁻⁵ or higher.

Furthermore, the invention provides a kit comprising the electrocompetent cell.

Also included are electrocompetent cells prepared from GB2005, GB05-red and GB05-dir cells.

In a third aspect, the present invention provides a method of introducing exogenous material into Gram-negative bacterial cells comprising growing Gram-negative bacterial cells in a medium, isolating the bacteria, and washing the bacteria under about 20 to about 30° C., thereby obtaining a electrocompetent cell, and electroporating the electrocompetent cell in the presence of the exogenous material.

In a fourth aspect, the present invention provides a method of transforming Gram-negative bacteria comprising growing the Gram-negative bacteria in a medium, isolating the cells and washing the cells under about 20 to about 30° C., and introducing nucleic acid molecules into the electrocompetent cells by electroporation.

In a fifth aspect, the present invention provides Gram-negative bacteria comprising nucleic acid molecules or other exogenous material as prepared by the present methods disclosed herein.

In a sixth aspect, the present invention provides a method of transforming Gram-negative bacteria comprising introducing nucleic acid molecules into the electrocompetent cells by electroporation under about 20 to 30° C.

In a seventh aspect, the present invention provides a kit comprising electrocompetent cells where the kit has a shipping or delivery temperature of higher than −20° C.

In an eighth aspect, the present invention provides a method of transforming Gram-negative bacteria comprising a) growing Gram-negative bacteria in a medium; b) isolating the bacteria and washing the bacteria with water or non-ionic liquid, thereby obtaining electrocompetent cells, wherein the washing in step b) takes place under 4° C.; and c) introducing nucleic acid molecules into the electrocompetent cells by electroporation under about 20 to about 30° C. Preferably, in step c) the cells are kept under about 20 to about 30° C. for 3 to 5 minutes prior to electroporation.

In a ninth aspect, the present invention provides a method of cloning of PCR products via linear to linear Red/ET homologous recombination.

In a tenth aspect, the present invention provides a method of cloning of genomic DNA fragments directly via linear to linear Red/ET homologous recombination where library construction and screening is not needed

The exact nature of this invention, as well as its advantages, will become apparent to a skilled person from the following description and examples. The present invention is thus not limited to the disclosed preferred embodiments or examples. A skilled person can readily adapt the teaching of the present invention to create other embodiments and applications.

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range. It includes, however, also the concrete number, e.g., about 20 includes 20.

The term “less than” or “more than” includes the concrete number. For example, less than 20 means less than or equal to. Similarly, more than or greater than means more than or equal to, or greater than or equal to, respectively.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

All publications and patents cited throughout the text of this specification (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

### BRIEF DESCRIPTION OF DRAWINGS

FIG. 1: Plasmid maps for pRK2-apra-km and pBC301.

FIG. 2.1: Different Gram-negative bacteria were made competent and transformed by electroporation using the present method (left panel) and conventional method (right panel), plated on antibiotic-containing plates.

FIG. 2.2: Transformation efficiency of Gram-negative bacterial strain Burkholderia glumae using the present method (right column) and conventional ice-cold method (left column).

FIG. 3: Effect of temperature shift on electrocompetent E. coli cell preparation. Cells were prepared and electroporated ice-cold or at room temperature.

FIG. 4: Effect of temperature shift on electrocompetent E. coli cell preparation. Transformation efficiency dramatically increased when competent cells were shifted from ice-cold to room temperature.

FIG. 5: Transformation results of electrocompetent E. coli cells stored under room temperature for different periods of time before electroporation.

FIG. 6: Transformation results of electrocompetent E. coli cells prepared at 15, 20, 22 and 24° C.

FIG. 7: Transformation results of electrocompetent E. coli cells prepared at 2, 15, 20, 22, 24, 26, 28, 30, 32, 34 and 37° C.

FIG. 8: Transformation results of electrocompetent E. coli cells taken from different growth phase (OD 600 is 0.4 (#1), about 1.2 (#2) and more than 1.8 (#3)) and prepared using present and conventional method.

FIG. 9: Transformation results of electrocompetent E. coli cells prepared without (#1) and with (#2) a recovery step.

FIG. 10: Transformation results of electrocompetent E. coli cells transformed with different plasmids.

FIG. 11: Transformation results of electrocompetent E. coli cells transformed with different plasmids.

FIG. 12: 12A illustrates linear to linear recombineering (LLHR) assay. 12B and 12C show the result of LLHR using electrocompetent cells prepared under RT or using conventional method.

FIG. 13: Diagram of LLHR using two homology arms to recombine two plasmids.

FIG. 14: LLHR results with or without carrier oligo using present or conventional methods are shown.

FIG. 15: Linear to circular recombination (LCHR). 15A illustrates LCHR assay. 15B and 15C show the result of LCHR using electrocompetent GB05-red cells prepared under RT or using conventional method. 15D shows the temperature shift increasing the transformation efficiency.

### DETAILED DESCRIPTION OF THE INVENTION

Routine procedures in biotechnological laboratories involve the use of various competent cells for cloning, propagation, and preparation of plasmid DNA, construction of genomic libraries and protein expressions. Competent cells possess the ability to take up exogenous material. They can be transformed, which means to be treated to take up nucleic acid molecules so that the genetic makeup of the cells is changed.

There are several methods for introducing exogenous material into bacteria. One of the first methods described was a chemical method described by Mandel and Higa (1970, Journal of Molecular Biology 53, 159) which teaches the steps of adding DNA to cells on ice in the presence of Ca²⁺ ions followed by a heat shock at 37° C. to 42° C. A more established method for preparing competent cells can be found in the Laboratory Manual by Sambrook et al. (1989, Molecular Cloning, 2nd Edition, 1.82). Sambrook et al describe a method where E. coli cells are grown in a suitable culture medium at 37° C., cooled on ice, separated from the medium by centrifugation, and resuspended in ice-cold 0.1M CaCl₂. After addition of cryoprotectant, the cell suspension is quickly frozen and kept at −70° C. for subsequent use in transformation experiments. As described in the laboratory manual, the cells as prepared above are thawed on ice and a small volume of DNA solution is added and mixed. The suspension is stored on ice for 30 minutes and then a heat-pulse at 42° C. is given. After chilling on ice, a volume of culture medium is added, and the cells are transferred to 37° C. to allow recovery. Then the transformed cells are plated out to allow identification of transformants. In general, chemically transforming the cells involves treating the cells with agents (such as calcium chloride or rubidium chloride) to induce the formation of pores in the cell wall, and incubating the treated cells with the nucleic acid.

Another method of introducing exogenous material into bacteria is electroporation. This has been described in Dower et al., Nucleic Acids Research, 16: 6127-6145 (1988); Taketo, Biochimica et Biophysica Acta, 949: 318-324 (1988); Chassy and Flickinger, FEMS Microbiology Letters, 44: 173-177 (1987). A typical electroporation method for bacterial cells is to grow bacteria in enriched media and to concentrate the bacteria by washing in water or 10% glycerol in water or non-conductive solution (Dower et al., 1988, U.S. Pat. No. 5,186,800, incorporated herein). As disclosed in U.S. Pat. No. 5,186,800, DNA is added to the cells and the cells are subjected to an electrical discharge, which temporarily disrupts the outer cell wall of the bacterial cells to allow the DNA to enter the cells. Another method is taught by U.S. Pat. No. 6,040,184 where E. coli cells are grown in approximately 12 liters of medium in a 15 liter fermenter. The fermenter is cooled to 4° C. When cells reach the desired density, the cells are concentrated by filtration. When the fluid level in the fermenter is reduced to 0.75 liters, buffer exchange is performed, running until 2 gallons of sterile 4° C. water has been exchanged. Another buffer exchange is then performed, running until 1 gallon of cold 15% glycerol has been exchanged. Cells treated in this manner are centrifuged gently (4000 rpm in a Sorvall swinging bucket rotor, 0° C., for 15 minutes) and resuspended in 35 ml of cold 15% glycerol. Electrocompetent cells prepared are then aliquoted for frozen storage or immediate use.

In developing and refining electroporation methodology, researchers have identified several factors that impact the efficiency. These factors include the electrical field strength, the pulse decay time, the pulse shape, the temperature in which the electroporation is conducted, the type of cell, the type of suspension buffer, and the concentration and size of the nucleic acid to be transferred (Andreason and Evans, (1988); Sambrook, et al (1989); Dower et al., (1988) and Taketo (1988)). For example, an improvement taught by U.S. Pat. No. 6,040,184 is to add one or more sugars or sugar aldoses to the competent cells prior to electroporation. It is taught that the sugars have a protective effect against electrical-treatment-related cell death. In another method, U.S. Pat. Appl. No. 2004/0209362 provides a strain of E. coli that has genetic modifications permitting enhanced survival of electrical treatment.

The inventors in the present invention have sought to improve the electroporation methodology by refining the preparation of electrocompetent cells. The present invention in one aspect provides improved methods of preparing electrocompetent cells from Gram-negative bacteria as described herein. The present invention is partly based on the surprising finding that electrocompetent cells can be prepared at an elevated temperature (elevated to room temperature). This is highly surprising because the conventional wisdom has been that electrocompetent Gram-negative bacteria must be strictly prepared under 4° C. and preferably at 0-2° C. In addition, the inventors have identified an optimal temperature range which leads to the improved performance of such electrocompetent cells. This surprising discovery permits the preparation and distribution of electrocompetent cells at a higher temperature as well as the simplification of the method which was not known as possible. By simply preparing the bacteria under about 20° C. to about 30° C., and especially under about 24° C. to about 28° C., the novel method unexpectedly serves to increase efficiency of electroporation.

The invention will be useful with Gram-negative bacteria which have previously been recognized as suitable hosts for receiving exogenous material.

As defined herein, the term “competent cells” refers to cells that are able to take up exogenous genetic material. Cells can be naturally competent or made competent by chemical means or by electroporation. “Electrocompetent cell” is defined herein as cells which are prepared for electroporation and when subjected to electroporation allows the entry of exogenous material (e.g., nucleic acid molecules) into the cell. As defined herein, “electroporation” means subjecting cells to an electric field such that an increase in the electrical conductivity and the permeability of the cell plasma membrane occur. An electrocompetent cell is typically prepared by growing cell cultures to a preselected cell density, harvesting the cells, and washing the cells to reduce the salts present so that when the cells are ultimately suspended in suspending medium, the electrical conductivity of the suspension will be low enough to prevent arcing.

In a first aspect, the present invention provides a method of preparing electrocompetent cells from Gram-negative bacteria, comprising:
a. growing Gram-negative bacterial cells in a medium,
b. isolating the cells and washing the cells with water or a non-ionic liquid,
c. optionally drying the cells, and
d. optionally storing the cells under about −80° C. to about 30° C.,
   wherein the washing in step b) takes place under about 20° C. to about 30° C.

The present invention provides in a second aspect an electrocompetent cell prepared by the present method.

A “cell” or a “bacterium” used in the present application refers to a Gram-negative bacterial cell.

As defined herein, Gram-negative bacteria refer to bacteria that do not retain the dark blue or violet by Gram staining. Gram-negative bacteria include, not limited to, the phyla Aquificae, Fusobacteria, Gemmatimonadetes, Nitrospirae, Proteobacteria, Spirochaetes, Synergistetes, Bacteroidetes and some members of Firmicutes. The bacteria is preferably Proteobacteria. All proteobacteria are Gram-negative, and examples include, but not limited to, Escherichia, Salmonella, Burkholderia, Pseudomonas, Agrobacterium, Photorhabdus, Xenorhabdus, Myxobacteria, Xanthomonas, Francisella, Agrobacterium, Helicobacter, Magnetospirillum, Rhodospirillum, Campylobacter, Rhizobium, Bordetella, Francisella, Shigella, Serratia, Brucellaceae, Bradyrhizobium, Acinetobacter, Alcaligenes, Brucella, Vibrio, Proteus, Yersinia, Pasteurella, Haemophilus, Zymomonas, Klebsiella, Erwinia, Legionella, Desulfovibrio, Bartonella, Azotobacteraceae, Rhizobiaceae, Nitrobacteraceae, Nitrobacter, Hydrogenophilaceae, Acetobacteraceae, Spirillaceae, Aeromonas. Other non-limiting examples of Gram-negative bacteria not belonging to proteobacteria are Treponema and Borrelia.

In a preferred embodiment, the Gram-negative bacterium is Escherichia, Salmonella, Burkholderia, Pseudomonas, Agrobacterium, Photorhabdus, Xenorhabdus or Myxobacterium. Most preferably, the Gram-negative bacterium is E. coli.

The current method is also applicable to different strains of a given Gram-negative bacterium. For example, applicable E. coli strains include HS996, BB4, BJ5183, K12, C600, DH5, DH5α, DH5α-E, DH5αMCR, DH5G, DH10, DH10B, DH10b/p3, DH10BAC, DY380, DY380-30C, GM2929, HB101, RR1, JV30, DH11S, DM1, DH10B/p3, DH5α5′lQ, DH5α5′, SCS1, Stab2, DH12S, XL1-Blue MRF, XL1-Blue MR, P2392, SCS1, SCS110, Stab2, SURE Strain, SURE 2 Strain, LE392, XL1-Blue, XL1-Blue MRF, XL1-BlueMR, XL2-Blue, YZ2005, AG1, JM101, JM103, JM109, JM110/SCS110, MC1061, NM514, NM522, NM554, TOPP Strains, Top10, ABLE Strains, MM294, XL1-Red, BL2I Strains, TK BI Strain, XL10-Gold, and derivatives thereof.

Information relating to the genotypes of these strains is available in the art. Preferably, the E. coli strain is HS996 and its derivatives, including GB2005. GB2005 is described in in Marcello Maresca et al 2010, Single stranded heteroduplex intermediates in λ Red homologus recombination, BMC Molecular Biology 11:54, as well as in WO 2011/154927. It has been surprisingly found that GB2005 is particularly suited for use in the present invention. In other preferred embodiments, the cells are GB2005-red and GB2005-dir, both also described in WO2011/154927. The genotype of GB2005 is: F⁻ mcrA Δ(mmr-hsdRMS-mcrBC) φ80dlacZΔM15 ΔlacX74 endA1 recA1 deoR Δ(ara, leu)7697 araD139 galU galK nupG rpsL fhuA λ⁻ recT, ybcC. The genotype of GB2005-red is: GB2005 with P_{BAD}-γβαA integrated into ybcC locus. The genotype of GB2005-dir is: GB2005 with P_{BAD}-ETγA integrated into ybcC locus.

As used herein, a “derivative” of a specified bacterium is a progeny or a recipient bacterium that contains genetic material obtained directly or indirectly from the specified bacterium. Such derivative bacterium may, for example, be formed by removing genetic material from a specified bacterium and subsequently introducing it into another bacterium (i.e., the progeny or other recipient bacterium) (e.g., via transformation, conjugation, electroporation transduction, etc.). Alternatively, such derivative bacterium may possess genetic material (produced synthetically, via cloning, via in vitro amplification, etc.) having the effective sequence of genetic material of the specified bacterium.

Growing Gram-Negative Bacteria

The first step of the present invention involves growing the Gram-negative bacteria in a medium. As defined herein, “growing” refers to enabling the proliferation of bacteria by providing required nutrients. The culturing and growth of the bacteria are well known in the art. For example, the user may refer to Ausubel et al 1981 (Current Protocols in Molecular Biology, John Wileys and Sons) and Nikoloff et al 1995 for growing E. coli. See Lin 1995 (Electrotransformation of Agrobacterum, in Methods in Molecular Biology, 47) for growing Agrobacterium. See O'Callaghan et al 1990 (High efficiency transformation of Salmonella typhimurium and Salmonella typhi by electroporation. Mol Gen Genet. 223(1):156-8) and Trevors 1990 (Electroporation and expression of plasmid pBR322 in Klebsiella aerogenes NCTC 418 and plasmid pRK2501 in Pseudomonas putida CYM 318. J Basic Microbiol.; 30(1):57-61) for growing Klebsiella; Mack et al 1996, (Transformation of Burkholderia pseudomallei by electroporation, Anal Biochem. 242(1):73-6) for growing Burkholderia; See Xu et al 1989 (Transformation of Xenorhabdus nematophilus. Appl Environ Microbiol.; 55(4): 806-12) for growing Xenorhabdus. Users can also consult Dower et al 1992 Protocols for the transformation of bacteria by electroporation and Trevors et al 1992, Electrotransformation of bacteria by plasmid DNA. Both articles are found in Guide to Electroporation and Electrofusion, Chang et al (eds.) Academic Press Inc. 485, San Diego, USA.

Growing may be performed first by streaking a drop of the selected bacteria stock on a favorable growth medium and incubating at a temperature favorable for growth. Once cultured, a single colony from the growth media can be selected to produce a starter culture of cells for the bacteria. As used herein, the term “medium” refers to a liquid or gelatinous substance containing nutrients which supports bacteria growth.

A starter culture of cells may be grown in a medium to produce large quantities of cells. This may be generated by incubating the cells, preferably from a single colony of the cell line, in a growth conducive medium at a favorable temperature for a period of time. A growth conducive medium may include a medium such as SOC medium. The incubation period may be for several hours, such as from 5 to 18 hours, preferably for about 12 to 18 hours, or overnight. During the incubation period, the medium is maintained at a temperature that is favorable to the growth. For many bacteria this is between about 20° C. to about 40° C. The growth may be further promoted by subjecting the medium to mixing, shaking or agitation, and preferably constant agitation. The bacteria in the cell culture solution may be cultured on a rotary shaker for several hours. For example, a culture of E. coli cells may be incubated with constant agitation on a shaker at about 180 rpm to about 250 rpm.

When the starter culture contains a desired concentration of the bacterial cells, it may be diluted and the cells further grown in growth conducive media. The starter solution may optionally be diluted to about 1:50 to about 1:100 with incubation media. SOB, SOC or LB medium can be used. The starter culture or diluted starter culture may be used to inoculate a freshly prepared growth conducive media at a favorable temperature range for the bacteria. For E. coli, a favorable growth temperature is between about 28° C. to about 40° C., preferably between about 28° C. to about 37° C., and most preferably at about 37° C.

Bacterial cells can be grown in a shake flask or fermenter. Culturing of cells by other means is well known by those skilled in the art. Shake flasks of any number of commercially available sizes and materials are suitable. For example, 1 L conical flasks with 100 mL of medium can be used for the culture. The actual conditions employed will of course depend on the bacteria being grown, flask size, temperature of incubation, and rate of shaking. Those skilled in the art are able to determine the optimal conditions for growing different bacterial cells, including growth conditions, culture age, and media. Teachings can be found in for example Nikoloff 1995. The incubation temperatures for growing the cells may vary from 3° C. to 42° C.

In one embodiment, cells may be grown in the presence of hyperosmotic salt concentrations in the medium. As described in U.S. Pat. Appl. No. 2004/0209362, those cells may have increased resistance to the trauma caused by electroporation. In practice, cells can be grown in standard rich growth media suitable for the selected species of bacteria (e.g., LB, SOB, SOC, Psi broth, TB, TY, etc.), supplemented with a hyperosmotic concentration of salt.

The culture is incubated until it reaches a desired cell concentration, which can be measured by the optical density of the culture. It is well known that bacteria live through four stages: lag phase, where growth is about to begin; log growth, where bacteria count increases exponentially; stationary growth, where as many bacteria die as reproduce; and endogenous or log death, where bacteria consume each other or when food stuff is scarce.

Typically, the bacteria are grown out to a preselected density where the cells are still rapidly dividing. The conventional wisdom is that the collection of rapidly growing cells is related to high transformation efficiencies. It has been known in the art that the highest transformation efficiencies are obtained when the cells are harvested in early to mid-log growth. For E. coli, it is known that when the cells reach stationary phase, the transformation efficiency will decline precipitously (Dower 1990, Electroporation of bacteria: a general approach to genetic transformation, in Genetic Engineering—Principles and Methods, 12, Plenum Publishing Corp., N.Y. 275). Generally, to reach mid-log phase bacteria are grown until an optical density OD at 600 nm of about 0.4 to 1.0 and more preferably about 0.6 to about 0.8 is reached

On the other hand, it is also known that cells harvested from early or mid-log phase are sensitive to various stresses, including osmotic and thermal stress, making their preservation by drying difficult (see Billi et al 2000 Engineering desiccation tolerance in Escherichia coli. Appl. Environ. Microbiol. 66:1680-1684). Electrocompetent cells are often produced in large quantities. Being able to use cells harvested from stationary phase would mean more efficient use of material for production, which is highly beneficial in large-scale production of electrocompetent cells.

Indeed, the inventors have found a method which allows the use of cells harvest from stationary phase to prepare electrocompetent cells. As used herein, “stationary phase” is reached when the bacteria is grown until an optical density at 600 nm of more than 1.6, such as more than 1.7, 1.8, 1.9, 2.0, 2.1, 2.2, 2.3, 2.4, 2.5, 2.6, 2.7, 2.8, 2.9, 3.0, 3.5, 4.0, 4.5, 5.0 or more.

Although in another embodiment, the Gram-negative bacteria is grown until an optical density at 600 nm of more than 0.4, such as 0.5, 0.6, 0.7, 0.8, 0.9, 1.0, 1.1, 1.2, 1.3, 1.4, 1.5, and 1.6.

Suitable cell densities at which cells are harvested can be determined without undue experimentation by one skilled in the art for a given species or strain of bacteria.

In one embodiment, a drop of glycerol stock of E. coli is streaked on a LB plate and incubated overnight at 37° C. for approximately 12-18 hours. A single colony from the plate is used to produce a starter of E. coli. This starter is used at a dilution of 1:100 to inoculate freshly prepared SOB or LB at a temperature of about 30° C. or about 37° C. Cells are cultured at approximately 30° C. on rotary shaker at a speed of about 180 to 250 rpm/min for 3 to 10 hours. The cells were collected upon reaching an OD600 of 1.0 to 2.0.

Isolating and Washing the Cells

It is known that electroporation is an ionic restricted physical process that requires a cell suspension of high resistance and very low conductivity for a high degree of success. During typical electrocompetent cell preparation, sodium chloride is normally used as a major ion source in the culture medium (for example about 1% in LB medium). As a result the salt has to be removed to ensure good electroporation results.

Thus, in a further step of the present method, bacterial cells reaching the determined density are isolated and washed to obtain electrocompetent cells. As used herein, “isolating” the cells refers to the removal of the cells from the media. This may be done, for example, by centrifugation or filtration, although other techniques to isolate the cells are known to those skilled in the art.

The present invention is characterized in that the isolated cells will be washed with water under room temperature. This means the chilling of the cells prior to or after the isolation to under 4° C. as required conventionally can be conveniently omitted.

Room temperature as used throughout the application means between about 20° C. to about 30° C., such as about 21° C. to about 29° C., such as about 22° C. to about 28° C., such as about 24° C. to about 28° C., including about 21, 22, 23, 24, 25, 26, 27, 28, or 29° C. “Washing” used herein means the application of a liquid to the cells for the purpose of removing residual salt from the cell. The term “salt” refers to any chemical compound that comprises an anion and a cation.

Washing is done by using water or non-ionic liquids defined herein as a solution with minimal or no ions. Non-ionic liquid may be non-polar such as glycerol. Glycerol helps pelleting the cells down in the process and is also a cyroprotectant that aids in preserving the cells if they are subsequently frozen for storage. Suitable liquids well known in the art are for example water (preferably sterile such as distilled), glycerol (about 5% to about 10%) or dimethyl sulfoxide (about 2% to about 15%). Non-ionic solutions are used for washing so that when the cells are suspended in the cuvette for electroporation and electricity is discharged into the host cells, little or no additional current than predicted is carried into the cell. The presence of ions in the suspension may result in additional current being carried into the cell which can lower the survival rate of the host cells.

In one preferred embodiment, the non-ionic solution has low conductivity. This is defined as a conductivity of 30 μs/cm or lower, such as 25 μs/cm, 20 μs/cm, 15 μs/cm, 12 μs/cm or lower.

The washing step is carried out by washing the cells with water or non-ionic liquid having a temperature of about 20° C. to about 30° C., such as about 24° C. to about 28° C., including about 21, 22, 23, 24, 25, 26, 27, 28, or 29° C. The cells are preferably washed more than 1 time, such as 2, 3, 4 times or more. Different non-ionic liquids can be used for separate washes if washed for more than once.

In one preferred embodiment, the step is carried out by washing the cells with water having a temperature of about 20° C. to about 30° C., such as about 24° C. to about 28° C., including about 21, 22, 23, 24, 25, 26, 27, 28, or 29° C. If the cells are immediately used for electroporation without drying, it is preferred that the cells are lastly washed with water. If the cells are to be dried, it is preferred that the cells are lastly washed with glycerol.

In another preferred embodiment, the step is carried out by washing the cells with glycerol, preferably glycerol water solutions, having a temperature of about 20° C. to about 30° C., such as about 24° C. to about 28° C., including about 21, 22, 23, 24, 25, 26, 27, 28, or 29° C.

The washing step may be performed by suspending the cells in the water or non-ionic liquid, pipetting the cells up and down, and isolating the cells from the non-ionic liquid. Isolation can be carried out under room temperature by any means known to a skilled person, including centrifugation, filtration or other means. As defined herein, the term “pipetting the cells” refers to using of any type of pipette (e.g., micropipette) to draw and release a sample of the non-ionic liquid which contains the cells. The electrocompetent cells can be immediately used for electroporation or stored. In one embodiment, the washing is carried out by washing the cell in a tube or microtube having room temperature.

Conventionally, after the cells reach the desired density the cells are kept under 4° C. or ice-cold. This requires that anything which comes in contact with the cells to be pre-chilled, including the non-ionic solution, rotor, centrifuge bottles or tubes, pipette tips or other devices and solutions which are used, sometimes even pre-chilled the day before. Since washing according to the present invention is not performed under low temperature, the chilling steps are advantageously eliminated. The present invention thus offers a simple and quick method for preparing electrocompetent cells.

Advantages

It has been surprisingly found that preparing electrocompetent Gram-negative bacteria at room temperature leads to one or more of the following improvements: 1. Easier—no preparation on wet ice and pre-chilling of equipment necessary. 2. Faster—since maintenance of lower temperature is not necessary, no waiting time for cooling down is needed 3. Higher Transformation Efficiency—the inventors have surprisingly found that the preparation at room temperature leads to the astonishing result of increased transformation efficiency. 4. No recovery step is needed, although may still be performed, after cells are electroporated. 5. Cells are more amenable to preservation by drying—the present method works well with cells harvested from stationary phase which are less fragile. 6. Efficient—use of cells harvested from stationary phase means more cells can be prepared at once. 7. Cheaper shipping or delivery costs—Delivery with dry ice is not necessary.

Drying the Cells

The present invention further comprises the step of optionally drying the electrocompetent cells. As used herein, “drying” refers to the reduction of the moisture content of the electrocompetent cells. Drying is preferably carried out under less than 30° C., such as 29, 28, 27, 26, 25, 24, 23, 22, 21, 20, 19, 18, 17, 16, 15, 14, 13, 12, 11, 10, 9, 8, 7, 6, 5, 4, 3, 2, 1, 0, −1, −2, −3, −4, −5, −6, −7, −8, −9, −10° C. or less and optionally under vacuum.

It is known that bacterial cells can be dried and sealed in containers to prevent ingress of water and thus allowing storage. Heckly (1961, Advances in Applied Microbiology, 3, pp 1-76) describes the application of freeze-drying and liquid-drying to the long-term preservation of bacteria. For free-drying and liquid drying users can also refer to Malik, K. A. & Claus, D., 1987, Biotechnology and Genetic Engineering Reviews, 5, pp 137-166).

Liquid-drying is a process by which water is removed from cells suspended in liquid (typically containing high concentrations of carbohydrate or substances that protect the cells during drying) and by which the cell suspension is exposed to a vacuum to achieve drying. Gas drying is a process by which water is removed from cells suspended in liquid (typically containing high concentrations of carbohydrate or substances that protect the cells during drying) by which the cell suspension is exposed to a dry gas atmosphere, typically air, nitrogen or argon gas to achieve drying. The atmosphere can be kept dry by exposure to a very dry dessicant, for example silica gel, in a sealed chamber. With time the suspension dries to such an extent that the product becomes substantially dry. Freeze-Drying is also called lyophilization in the art, which is drying from the frozen state whereby ice and/or moisture is removed from frozen cells by sublimation under vacuum at low, subzero temperatures, typically yielding a cake. Air-drying, vacuum-drying, oven-drying, spray-drying, flash-drying, fluid bed-drying, and controlled atmosphere drying are described in U.S. Pat. No. 5,728,574; U.S. Pat. No. 5,733,774; U.S. Pat. No. 5,200,399; U.S. Pat. No. 5,340,592; and U.S. Pat. No. 4,797,364. Convective drying is described in Lievense, L. C. and van't Riet, K., 1994, Advances in Biochemical Engineering/Biotechnology, 51, pp 71-89. Spray-drying is described in Franks and Hatley, 1992, EP0520748 and granulation of organisms is described in Muller, H., 1978, EP493761. Other methods of preparing dried bacterial cells are also known in the art, e.g., Annear 1966 (Nature, 211:5050, 761) and WO 98/35018.

In one embodiment, cells are dried under vacuum, and optionally at non-atmospheric pressure, e.g., 1000-4000 mtorr. Jesse and Bloom (1997) Patent Application WO 98/35018 describe the removal of the ice of frozen suspensions of competent cells in a freeze-drier is by sublimation under vacuum.

Preferably, the cells are dried such that the cells contain at least 20% less moisture, for example 25%, 30%, 35%, 40%, 45%, 415%, 14%, 13%, 12%, 11%, 10%, 9%, 8%, 7%, 6%, 5%, 4%, 3%, 2% and 1%. Moisture can be measured by any method in the art including weight loss on drying.

Dried electrocompetent cells can be re-hydrated by the addition of suitable liquid for electroporation. Cells can for example be rehydrated in water, 4-5% trehalose solution or 10% glycerol.

Storing the Cells

The method further comprises the step of optionally storing the cells under −80° C. to 30° C. after the cells are washed or optionally dried. Cells can be stored frozen without prior drying step, for example by snap freezing aliquots, e.g., 200 μl on a dry ice-ethanol bath.

As defined herein, “storing” refers to keeping the cells under a suitable condition which keeps the cells viable. The conditions for storing electrocompetent cells are known in the art and described in the references cited in this application. Most commonly, 10% glycerol is used for cell storage. To date electrocompetent cells are stored at temperatures under −70° C. or below. For manufacturers of commercial competent cells, this requires a series of ultra-cold storage and transportation operations, including shipping on dry ice. With the present invention, it is not necessary to ship the electrocompetent cells prepared herein on dry ice although it is also possible. The delivery can be done at a higher temperature by using wet ice or gel ice or any other means which keeps the temperature at higher than −20° C. Cooling method are known in the art including liquid nitrogen, gel, dry ice, wet ice, ice cartridge or others known in the art.

The industry can thus greatly benefit from the present invention by reducing the costs associated with the maintenance of the low temperature condition required for cell preparation and storage.

If stored, it is preferred that the cells were lastly washed with 10% glycerol and dried.

In one embodiment, the cells are stored under about 0° C. to about 30° C., for example about 0° C. to about 25° C., about 0° C. to about 20° C., about 0° C. to about 10° C., about 0° C. to about 5° C. Such cells are therefore not thawed prior to electroporation.

In another embodiment, the cells are stored under about −80° C. to about 0° C., such as about −70° C. to about 0° C., about −60° C. to about 0° C., about −50° C. to about 0° C., about −40° C. to about 0° C., about −30° C. to about 0° C., about −20° C. to about 0° C., about −10° C. to about 0° C., about −5° C. to about 0° C.

In one preferred embodiment, the cells are stored under about −20° C. to about 10° C., such −15° C., −10° C., −5° C., 0° C., 1° C., 2° C., 3° C., 4° C., 5° C. and 10° C.

Electroporation

In another aspect, the present invention provides a method of introducing exogenous material into Gram-negative bacterial cells comprising:
a. growing Gram-negative bacterial cells in a medium,
b. isolating the cells and washing the cells with water or a non-ionic liquid,
c. optionally drying the cells,
d. optionally storing the cells under about −80° C. to about 30° C., and
e. introducing exogenous material into the cells by electroporation,
wherein the washing in step b) takes place under about 20° C. to about 30° C.

As used herein, the term “exogenous material” refers to material which does not originate from the bacteria to which the material is to be introduced. The material can be any type of molecules such as nucleic acids, polypeptides, carbohydrates, drugs, viruses, plasmids, phagemids, dyes, DNA, RNA, toxins, proteins, enzymes, antibodies, hormones, labeled nucleotides, amino acids or any other substances. Preferably the exogenous material is a nucleic acid molecule.

In another aspect, the present invention provides a method of transforming a Gram-negative bacterial cells comprising:
a. growing Gram-negative bacterial cells in a medium,
b. isolating the cells and washing the cells with water or a non-ionic liquid,
c. optionally drying the cells, and
d. optionally storing the cells under about −80° C. to about 30° C., and
e. introducing nucleic acid molecules into the electrocompetent cells by electroporation,
wherein the washing in step b) takes place under about 20° C. to about 30° C.

In a preferred embodiment, step e) also takes place at room temperature (i.e., under about 20° C. to about 30° C.).

In a further aspect, the present invention provides Gram-negative bacterial cell comprising exogenous material or nucleic acid molecules prepared by these methods.

As used to herein, nucleic acid molecule includes any nucleic acid molecule that is naturally occurring or synthetic. Such nucleic acid molecules can be DNA molecules or RNA molecules, including antisense RNA of any size, from any source, including DNA from viral, prokaryotic, and eukaryotic organisms, and also DNA/RNA hybrids A nucleic acid molecule can be in any form, including, but not limited to, linear or circular, and single or double stranded. Non-limiting examples of DNA molecules include plasmids, vectors, and expression vectors. Nucleic acid molecules may encode functional or non-functional proteins, The nucleic acid molecule is not limited to any size and may range from about 1 kb to about 500 kb or more, for example 1, 2, 3, 4, 5, 10, 20, 25, 30, 40, 50, 70, 90, 100, 150, 200, 300, 400 kb.

The cells DH5G, GB2005, GB2005-dir and GB05-red were found to have high ability to take up nucleic acid molecules of varying size ranges and for recombineering and are thus the preferred Gram-negative bacterium in one aspect of the present invention.

Electrocompetent cells prepared herein are electroporated for the introduction of exogenous material into the cells. Electroporation is the phenomenon that makes cell membranes permeable by exposing them to electric field. It is a well-known technique described in many references cited herein. This technique is based upon the original observation by Zimmerman et al. (J. Membr. Biol. 67: 165-82 (1983)) that high-voltage electric pulses can induce cell plasma membranes to fuse. Subsequently, it was found that when subjected to electric shock (typically a brief exposure to a voltage gradient of 4000-16000 V/cm) the bacteria take up exogenous DNA from the suspending solution, apparently through holes momentarily created in the plasma membrane. A proportion of these bacteria become stably transformed and can be selected if a suitable marker gene is carried on the transforming DNA (Newman et al., Mol. Gen. Genetics 197: 195-204 (1982)).

Methods and material for electroporating a bacterial cell are well known and described in the literature. For example, electroporation is described in U.S. Pat. No. 5,186,800, U.S. Pat. No. 6,338,965, U.S. Pat. No. 4,910,140, U.S. Pat. No. 5,964,726, U.S. Pat. No. 5,879,891, U.S. Pat. No. 6,586,249, Andreason and Evans, (Analytical Biochemistry, 180: 269-275 (1988)); Sambrook et al. (1989), Taketo (1988), Hanahan et al., Plasmid Transformation of Escherichia coli and Other Bacteria, Methods in Enzymology, V. 204, pp. 63-113 (1991), Dower et al. (1988), Potter, H., Electroporation in Biology: Methods, Applications, and Instrumentation, Analytical Biochemisty, V. 174, No. 2, pp. 361-373 (1988), Andreason and Evans, Biotechniques, 6: 650-660 (1988).

Commercially available electroporators include GENE PULSER® Xcell microbial system, the GENE PULSER® Xcell eukaryotic system, the GENE PULSER® Xcell total system, and the MICROPULSER® Electroporator, all of Bio-Rad Laboratories, Hercules, Calif., USA, the EPPENDORF® Electroporator 2510, the MULTIPORATOR® of Brinkmann Industries, Inc., Westbury, N.Y., USA, the ECM® 2001, ECM® 399, ECM® 630, and ECM® 830 Electroporator Systems, all of Harvard Apparatus Inc., BTX Instrument Division, Holliston, Mass., USA, the NUCLEOFECTOR™ Device of Amaxa Biosystems, Gaithersburg, Md., USA, the CELLJECT UNO, CELLJECT DUO, and CELLJECT PRO, all of Thermo Electron Corporation, Gormley, Ontario, Canada, and THE CLONING GUNTM (BactoZapper™) and THE CLONING GUN™ (MammoZapper™) of Tritech Research, Inc., Los Angeles, Calif., USA.

Conventional electroporation protocols require frozen electrocompetent cells to be thawed and transferred to an electroporation cuvette. Exogenous material is typically washed and added to the cell with suitable suspension solution into the cuvette. The cuvette contains electrical connections necessary for passing a current through the cell suspension. Suitable suspension solutions used are substantially non-ionic solutions and are known in the art. In general, a small volume of the high density cell suspension is introduced to the sample holding region of a cuvette so that the volume is contained substantially entirely between the electrodes. Typically, the total volume of the cell suspension will be in the range from about 5 to 400 μL, more typically being in the range from about 10 to 100 μL, and preferably below about 50 μL. Concentration of the cell suspension useful for electroporation is generally about 5×10⁹ to 1×10¹¹ cells/ml, preferably about 1×10¹⁰ to 5×10¹⁰ cells/ml.

According to the present invention, the electroporation of the electrocompetent cells is performed under room temperature. This means the cuvette does not have to be chilled as required conventionally.

In a preferred embodiment, the electroporation is carried out under room temperature i.e.

about 20° C. to about 30° C., such as about 24° C. to about 28° C., including about 21, 22, 23, 24, 25, 26, 27, 28, or 29° C. The inventors have surprisingly found that electroporation under room temperature also increases transformation efficiency even for cells that are prepared using the traditional method (under 4° C.). This is for example demonstrated by example 19.4 and 24. Accordingly, in another aspect the present invention includes a method of transforming Gram-negative bacteria comprising introducing nucleic acid molecules into the electrocompetent cells by electroporation under about 20 to about 30° C. The present invention provides a way of increasing transformation efficiency of cells prepared by conventional method (cold method) by elevating the cell temperature for electroporation. The cells, if prepared using traditional method (under 4° C.), can be left at room temperature for more than 3 minutes, such as 3, 4, 5, 6, 7, 8, 9, 10 minutes, and most preferably for 3-5 minutes before electroporation at room temperature. This finding is contrary to the pervasive opinion that electroporation should be performed at low temperature.

After electroporation the cells are conventionally subjected to a “recovery step” (Dower et. al., 1988 Nucleic Acids Res. 16: 6127-6145). In this step the cells are cultured in a nutrient medium to allow the electroporated cells to recover their physiological function. In the regard the inventors have found that this step can be omitted. The recovery step is defined here as incubating the electroporated cells in a non-selective medium (e.g., SOC, LB) for more than 30 minutes, such as 1-2 hours.

In one preferred embodiment, the electroporation step further comprises selecting bacterial cell which are successfully transformed. This can be done by incorporating a selective marker (e.g., an antibiotic resistance gene) in the molecule to be introduced. The marker allows selection of transformed cells if the corresponding antibiotic is used in the medium. After the cells have been electroporated with the DNA molecule, the transformed cells can be directly transferred to the selective medium without recovery. This means the present invention has the advantage of shortened electroporation process.

In a further aspect, the present invention provides a method of transforming Gram-negative bacteria comprising: a) growing Gram-negative bacteria in a medium, b) isolating the bacteria and washing the bacteria with water or non-ionic liquid, thereby obtaining electrocompetent cells, wherein the washing in step b) takes place under 4° C. (for example at −2, −1, 0, 1, 2, 3° C.); and c) introducing nucleic acid molecules into the electrocompetent cells by electroporation under about 20 to about 30° C. In a preferred embodiment, the cells are stored or kept under about 20 to about 30° C. for 3 to 5 minutes prior to electroporation, for example by leaving the cells under room temperature for 3 minutes.

Transformation Efficiency and Absolute Transformation Efficiency

As will be appreciated by a skilled person reading the content as disclosed herein, the present invention is particularly useful for increasing the transformation efficiency of an electrocompetent cell. For the purpose of the present invention, “transformation efficiency” is determined using the given transformation reaction described in example 22. “Absolute transformation efficiency” is calculated by dividing the total number of transformants (with selection pressure) by the total number cells (without selection pressure) Electrocompetent cells prepared herein has. In some embodiments, an increased absolute transformation efficiency of at least 7×10⁻⁶, such as at least 8×10⁻⁶, at least 9×10⁻⁶, at least 1×10⁻⁵, at least 2×10⁻⁵, at least 3×10⁻⁵, at least 4×10⁻⁵, at least 5×10⁻⁵, at least 6×10⁻⁵, at least 7×10⁻⁵, at least 8×10⁻⁵, at least 9×10⁻⁵, at least 1×10⁻⁴, at least 2×10⁻⁴, at least 3×10⁻⁴, at least 4×10⁻⁴, at least 5×10⁻⁴, at least 6×10⁻⁴, at least 7×10⁻⁴, at least 8×10⁻⁴, at least 9×10⁻⁴, at least 1×10⁻³ or higher.

The term “increased,” “improved” or “higher” transformation efficiency refers to an “increase” in the property of a given cell strain to generate more transformed cells per unit of nucleic acid than a reference population of cells. The reference population is a population of cells of the same genotype that have not been treated in a manner that effect increased transformation efficiency (i.e., not prepared at room temperature). An “increase” as the term is used herein is at least 3%, preferably at least 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 60%, 70%, 80%, 90%, 100% (i.e., two-fold), 3, 4, 5, 6, 7, 8, 9, 10, 20, 30, 50, 100-fold or more relative to the transformation efficiency of the reference population.

Kit

In another aspect of the invention a kit comprising the prepared electrocompetent cells is provided. The packaging material of the kit can contain one or more articles for maintaining the electrocompetent cells between −80° C. to 30° C. Preferably, the packaging material maintains the cells at −20° C. to 10° C., such −15° C., −10° C., −5° C., 0° C., 1° C., 2° C., 3° C., 4° C., 5° C. and 10° C. for delivery. Any article known in the art for maintaining packaged goods at this temperature range can be used.

In yet another aspect, the present invention provides a kit comprising electrocompetent cells where the kit has a delivery temperature of higher than −20° C., such −15, −14, −13, −12, −11, −10, −9, −8, −7, −6, −5, −4, −3, −2, −1, 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30° C. As defined herein, delivery temperature refers to the temperature of the electrocompetent cells during the time of delivery or shipping.

Preferably, the kit comprises electrocompetent cells derived from GB2005, GB2006, GB2008 and GB2012 cells. The kit preferably includes a control plasmid or control BAC for testing electroporation efficiency.

The kit may be useful for producing a desired protein by transforming the electrocompetent cell of the invention with a DNA molecule encoding said desired protein. Transformed cells may be selected according to techniques well known in the art including, for example, selection for marker genes on the DNA molecule (e.g., antibiotic resistance genes). After the transformed cell has been selected, the cell may then be cultured according to well-known techniques in a growth conducive medium. Upon culturing the cell under appropriate conditions the cell is capable of producing the desired protein. The desired protein may then be isolated and purifed by well-known protein purification techniques.

The present invention is particularly useful for recombineering. Recombineering is a DNA engineering technology via homologous recombination in vivo mediated by RecET or lambda Red Gam Beta Alpha (red gba) (WO/2011/154927 and Fu et al. Enhanced direct DNA cloning by full length RecE and its application to secondary metabolites. Nature Biotechnology. Nature Biotechnology 30(5):440-446 (2012)). One of the limiting factors for recombineering is transformation efficiency because both molecules (donor and recipient) must meet each other in one cell by electroporation. The inventors have found that electrocompetent cells prepared the present method have dramatically improved transformation efficiency and also recombineering efficiency (see example 19). The present invention is therefore directed to a recombineering kit comprising an electrocompetent cell prepared by the presently disclosed method. The kit preferably comprises electrocompetent cells prepared from GB2012, GB2005, GB05-dir or GB05-red cells and optionally one or more control plasmids. GB2005 can be constructed from DH10B by deletion of fhuA, ybcC and recET22,49. GB05-dir can be constructed by integrating the P_{BAD}-ETgA operon, into the ybcC locus in GB2005. GB05-red can be constructed from GB2005 by insertion of the P_{BAD}-gbaA cassette at the ybcC locus22,49.

Also provided herein is a kit for PCR product cloning via linear to linear recombination (LLHR). The inventors have found that the homology arms for LLHR can be reduced to 12 bp or less. This makes the PCR product cloning easy and cheap. Current commercially available PCR product cloning kits are expensive and difficult to handle due to the in vitro reactions. A kit for PCR cloning preferably comprises electrocompetent GB2012, GB2005, GB05-dir or GB05-red cells and optionally additional oligonucleotides. Additionally the kit may also contain material for control experiment for LLHR (linear vector and linear PCR product), and linear cloning vectors.

Also provided herein is a kit for direct cloning. Direct cloning the gene fragments from chromosomal DNA pool without DNA library construction and screening is the ideal tool for many laboratories. WO2011/154927 describes the use of single stranded oligonucleotides (carrier oligos) to improve the efficiency of direct cloning. This kit may include electrocompetent GB2012, GB2005, GB05-dir or GB05-red cells and optionally additional single stranded oligonucleotides and control materials The single stranded oligonucleotides include any oligonucleotides with 10 nt-100 nt with average GC content but no homology to donor or recipient or host genome. The present kit allows efficient and routinely workable direct cloning.

Having now generally described the invention, the invention will be more readily understood through reference to the following examples, which are provided by way of illustration and are not intended to be limiting of the present invention. All patents, patent applications and publications mentioned herein are incorporated by reference in their entirety.

### EXAMPLES

### Example 1

### Preparation of Electrocompetent Cell from Agrobacterium

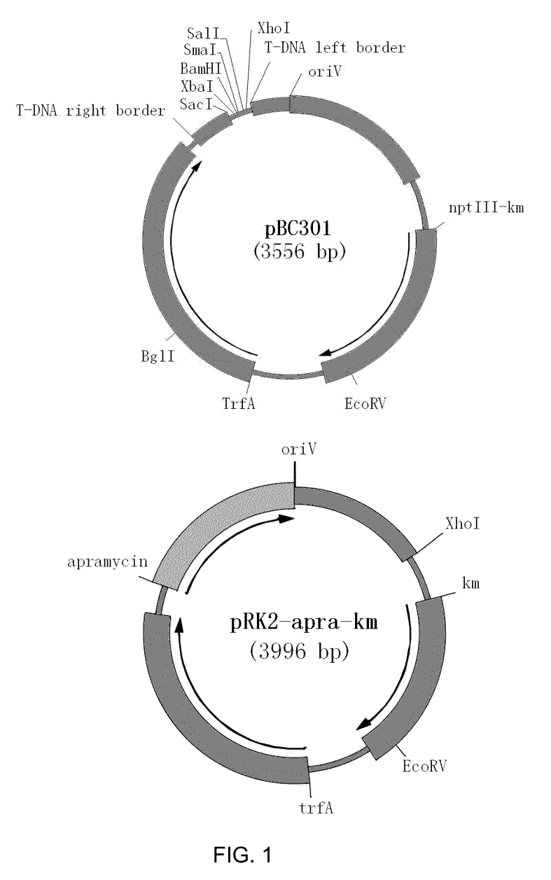

Agrobacterium were made electrocompetent and subsequently electroporated for transformation. An oriV or RK2 origin plasmid pRK2-apra-km (FIG. 1, bottom), which was constructed based on plasmid named pBC301 (Xiang et al., A mini binary vector series for plant transformation. Plant Mol Biol. 1999; 40(4):711-7; Yang et al. Functional Modulation of the Geminivirus AL2 Transcription Factor and Silencing Suppressor by Self-Interaction. J Virol. 2007; 81(21):11972-11981) was used for transformation. pRK2-apra-km contains RK2 (or oriV) origin, trfA—replicon gene, apramycin resistant gene and kanamycin resistant gene and is able to replicate in most of bacterial strains.

The preparation of electrocompetent cells was carried out according to the present invention under room temperature as well as under 4° C. for comparison. The protocol is as follows.

1.1 Preparation of Electrocompetent Cell Using Conventional Method

The following protocol was used:

Culture the bacterial strain overnight in LB medium. Take 50 ul of overnight culture into 1.4 ml LB medium and culture them at 30° C. with shaking at 900 rpm in an Eppendorf ThermoMixer. Spin down the bacterial cells when OD600 is about 0.7 at 9000 rpm in an Eppendorf centrifuge (4° C.). Discard the supernatant and resuspend the cells by dH₂O (ice-cold). Spin down at 9000 rpm in an Eppendorf bench centrifuge (4° C.). Repeat the wash step once. Resuspend the bacterial cells in about 30 μl of ice-cold dH₂O and place the cells on ice.

1.2 Preparation of Electrocompetent Cell Using Present Invention

The following protocol was used:

Culture the bacterial strain overnight in LB medium. Take 50 μl of overnight culture into 1.4 ml LB medium and culture them at 30° C. with shaking at 900 rpm in an Eppendorf ThermoMixer. Spin down the bacterial cells when OD600 is about 0.7 at 9000 rpm in an Eppendorf centrifuge (24° C.). Discard the supernatant and resuspend the cells by dH₂O (24° C.). Spin down at 9000 rpm in an Eppendorf bench centrifuge (24° C.). Repeat the wash step once. Resuspend the bacterial cells in about 30 μl of dH₂O (24° C.) and place the cells at 24° C.

The cells prepared from Example 1.1 using conventional method is then electroporated under ice-cold condition using the protocol as described in Example 1.3. The cells prepared from Example 1.2 using the present invention are electroporated under 24° C. using the protocol as described in Example 1.4.

1.3 Electroporation Using Conventional Method

The following protocol was used:

Add 2 μl of pRK2-apra-km plasmid DNA (0.3 μg) into the electrocompetent bacterial cells. Transfer the DNA-cell mixture into pre-chilled 1 mm-gap electroporation cuvette (ice-cold). Electroporate the DNA-cell mixture at 1250 volts. Add 1 ml of LB medium to resuspend the transformed cells. Incubate the cells at 30° C. for 1.5 hours to recover. Plate 100 μl of cells on kanamycin-containing LB plates.

1.4 Preparation of Electrocompetent Cell Using Present Invention

The following protocol was used:

Add 2 μl of pRK2-apra-km plasmid DNA (0.3 μg) into the electrocompetent bacterial cells. Transfer the DNA-cell mixture into 1 mm-gap electroporation cuvette (24° C.). Electroporate the DNA-cell mixture at 1250 volts. Add 1 ml of LB medium to resuspend the transformed cells. Incubate the cells at 30° C. for 1.5 hours to recover. Plate 100 μl of cells on kanamycin-containing LB plates.

### Example 2

### Preparation of Electrocompetent Cell from Burkholderia

Burkholderia thailandensis were made electrocompetent and subsequently electroporated for transformation. The protocols used were as described in Example 1.

### Example 3

### Preparation of Electrocompetent Cell from Photorhabdus

Photorhabdus luminescens were made electrocompetent and subsequently electroporated for transformation. The protocols used were as described in Example 1, with the exception that the bacterial cells prepared using conventional method were lysed after cold-water washing, but cells prepared using present method were not.

### Example 4

### Preparation of Electrocompetent Cell from Pseudomonas

Pseudomonas putida were made electrocompetent and subsequently electroporated for transformation. The protocols used were as described in Example 1

### Example 5

### Preparation of Electrocompetent Cell from Xenrohabdus

Xenrohabdus szentirmaii were made electrocompetent and subsequently electroporated for transformation. The protocols used were as described in Example 1

### Results from Example 1-5

The procedures in Example 1-5 were repeated twice each. Table 1 below shows the average colonies numbers appeared on the kanamycin containing LB plates. Clearly, by preparing the cell at room temperature has great impact on increasing transformation efficiency and up to more than 100 folds.

TABLE 1

| Gram-negative Bacterium | Conventional Method | Present method |
|---|---|---|
| Agrobacterium tumefaciens | 404 | 2260 |
| Burkholderia thailandensis | 56 | 1470 |
| Photorhabdus luminescens | 3 | 551 |
| Pseudomonas putida | 2000 | 21600 |
| Xenrohabdus szentirmaii | 47 | 2515 |

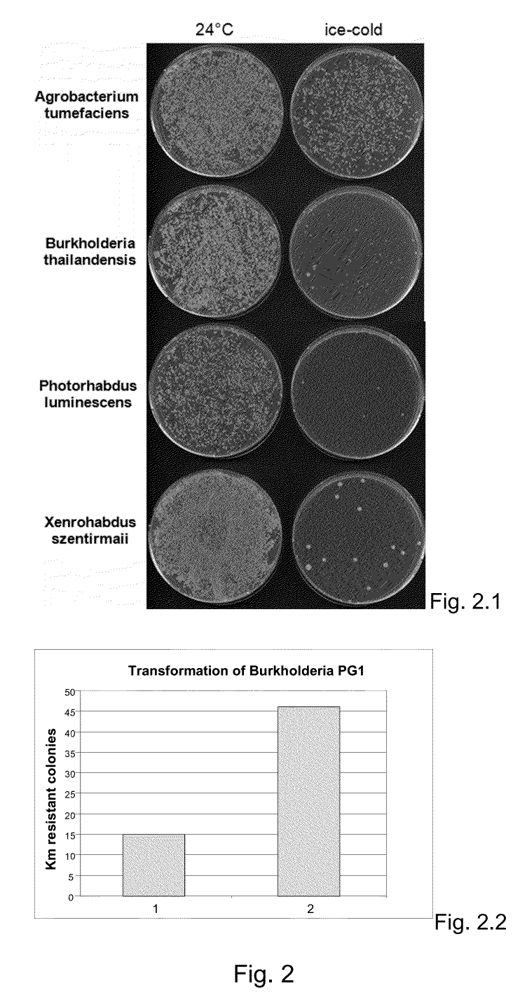

FIG. 2.1 shows the colonies of transformed Agrobacterium tumefaciens, Burkholderia thailandensis, Photorhabdus luminescens, and Xenrohabdus szentirmaii. Plates for Pseudomonas putida were too full and not shown.

### Example 6

### Preparation of Electrocompetent Cell from Burkholderia

Burkholderia glumae were made electrocompetent and subsequently electroporated for transformation. The protocols used were as described in Example 1. The Burkholderia strain PG1 is an industrial strain for detergent lipidase production and can be used for heterologous expression of PKS/NRPS gene clusters. pRK2-apra-km plasmid as described in Example 1 was used for transformation. The transformants are Km resistant. FIG. 2.2 shows when PG1 electrocompetent cells were prepared at room temperature, the electroporation efficiency is 3 times better than the cells prepared on ice.

### Example 7

### Preparation of Electrocompetent Cell from Escherichia

E. coli DH10B strain is grown in LB broth at 37° C. Inoculate 1 liter of autoclaved LB with 25 micro liter of an overnight-saturated culture of E. coli. Grow the cells to OD₆₀₀ at 1.5 or higher such as 2.0 while shaking at 37° C. Centrifuge in 4000×g for 15 minutes. Discard the supernatant. Resuspend the pellet in 1 L 24-28° C. H₂O and centrifuge as above. Resuspend again in 0.5 L 24-28° C. H₂O and centrifuge as above. Resuspend in 20 ml of 24-28° C., sterile 10% glycerol in H₂O and transfer to a 50-ml disposable Falcon tube. Centrifuge 3000 rpm under 24-28° C., 15 minutes. Resuspend in 3 ml of 24-28° C. sterile 10% glycerol.

Electroporations are carried in 2-mm gap cuvettes with an Eppendorf Multiporator at a setting of 2500 volts, generating a time constant of approximately 5 ms. Mix 1-2 microliters of DNA in TE (10 mM Tris-HCl, pH 7.8 and 1 mM EDTA) with the cells. Use 100 pg of supercoiled plasmid DNA as a positive control, and 0.5-1.0 ng (of vector) for a ligated construct. Set the Eppendorf Multiporator apparatus for 25 microFarad. Set the Pulse Controller to 2500 volts. Set the Gene Pulser to 2.50 kV for 2-mm cuvettes (or 1.2-1.8 kV for 1-mm gap cuvettes). Transfer the DNA-cell mixture to the bottom of an electroporation cuvette under room temperature. Place in cuvette chamber slide, and push the slide into the chamber to make contact with the electrodes. Pulse once per sample. The time constant is between 4-5 milliseconds. Remove the cuvette and immediately add 1 ml of L-broth and resuspend the cells. Transfer the cell suspension to a 1.5 ml polypropylene tube. No incubation is carried out. Plate cells on agar media with antibiotics, as needed.

### Example 8

### Preparation of Electrocompetent Cell from Escherichia

Inoculate 25 ml LB+antibiotic with a single E. coli colony and then inoculate 1 L LB+antibiotic with this 25 micro liter culture. Grow cells (37° C. and shaking) to OD 600 at 1.0-2.0. Spin down cells 15 min. (4K rpm) at 20-30° C. Remove as much supernatant as possible. Suspend cells in 1 L 20-30° C. H₂O. Spin down cells as before. Suspend cells in 0.5 L 20-30° C. H₂O and spin down again. Suspend cells in 20 ml 20-30° C. sterile 10% glycerol. Freeze 125 μl aliquots on dry ice. Pack the cells in sealed tub with wet ice for shipping.

### Example 9

### Preparation of Electrocompetent Escherichia and Electroporation

E. coli strain GB2005 (also called GB05) was made electrocompetent and subsequently electroporated for transformation. GB2005 is a derivative of DH10B, which is the optimal host for plasmid propagation and transformation (Fu et al. A recombineering pipeline to make conditional targeting constructs. Methods Enzymol. 477, 125-144 (2010); Fu, J. et al. Efficient transfer of two large secondary metabolite pathway gene clusters into heterologous hosts by transposition. Nucleic Acids Res. 36, e113 (2008)). Both the conventional method and current method were carried out. A large plasmid pGB-amp-Ptet-plu1880 (27.8 kb) (Fu et al. Enhanced direct DNA cloning by full length RecE and its application to bioprospecting for secondary metabolites. Nature Biotechnology 30(5):440-446 (2012)) was used for transformation using the following protocols.

9.1 Preparation Using Conventional Method

Preparation for the experiment: Cool an Eppendorf centrifuge to 2° C. or place centrifuge in a cold room. Cool down dH₂O on ice for at least 2 hours or take cooled dH₂O from fridge and place on ice. Place electroporation cuvettes and plasmid DNA on ice. Make a hole in the lid of an Eppendorf tube and inoculate 35 μl of GB2005 overnight culture into 1.4 ml LB medium. Incubate the tube in a heating block at 37° C. with shaking for 2 hrs and 45 min. GB2005 strain was cultured at 37° C. till OD600 to about 0.4-0.6 Spin down the cells at 9,000 rpm speed for 30 sec in an Eppendorf centrifuge at 2° C. or in a cold room. Discard the supernatant and place the tube on ice. On ice, resuspend the cells in 1.0 ml of ice-cooled dH₂O. Spin down the cells at 9,400 rpm speed for 30 sec. in an Eppendorf centrifuge at 2° C. or in a cold room and discard the supernatant. On ice, resuspend the cells in 1.0 ml of ice-cooled dH₂O. Spin down the cells at 9,400 rpm speed for 30 sec. in an Eppendorf centrifuge at 2° C. or in a cold room. Discard the supernatant using a 1 ml pipette leaving around 20-30 μl of solution or discard the supernatant by swinging the tube and leave around 20-30 ul. Add less than 4 μl of plasmid DNA and place mixture into an ice-cooled electroporation cuvette (1 mm gap) on ice. Using an Eppendorf electroporator, electroporate the cells at 1300V. Add 1 ml of LB medium into the cuvette and transfer back into the Eppendorf tube. Plate the cells on LB agar plates containing suitable antibiotic after 70 minutes recovery at 37° C. with shaking. Incubate the plates at 37° C. overnight.

9.2 Preparation Using Present Method

Make a hole in the lid of an Eppendorf tube and inoculate 35 μl of GB2005 cells overnight culture into 1.4 ml LB medium. Incubate the tube in a heating block at 37° C. with shaking for 2 hrs and 45 min. GB2005 strain was cultured at 37° C. till OD600 to about 0.4-0.6. Spin down the cells at 9,000 rpm speed for 30 sec in an Eppendorf centrifuge at room temperature (RT) such as 22° C.-28° C. Discard the supernatant and resuspend the cells in 1.0 ml RT dH₂O. Spin down the cells at 9,400 rpm speed for 30 sec. in an Eppendorf centrifuge at RT and discard the supernatant. Resuspend the cells in 1.0 ml of RT dH₂O. Spin down the cells at 9,400 rpm speed for 30 sec. in an Eppendorf centrifuge at RT. Discard the supernatant using a 1 ml pipette leaving around 20-30 μl of solution or discard the supernatant by swinging the tube and leave around 20-30 μl. Add less than 4 ul of plasmid DNA and place mixture into an RT electroporation cuvette (1 mm gap). Using an Eppendorf electroporator electroporate the cells at 1300V. Add 1 ml of LB medium into the cuvette and transfer back into the Eppendorf tube. Plate the cells on LB agar plates containing suitable antibiotic after 70 minutes recovery at 37° C. with shaking. Incubate the plates at 37° C. overnight.

### Example 10

### Effect of Temperature Shift

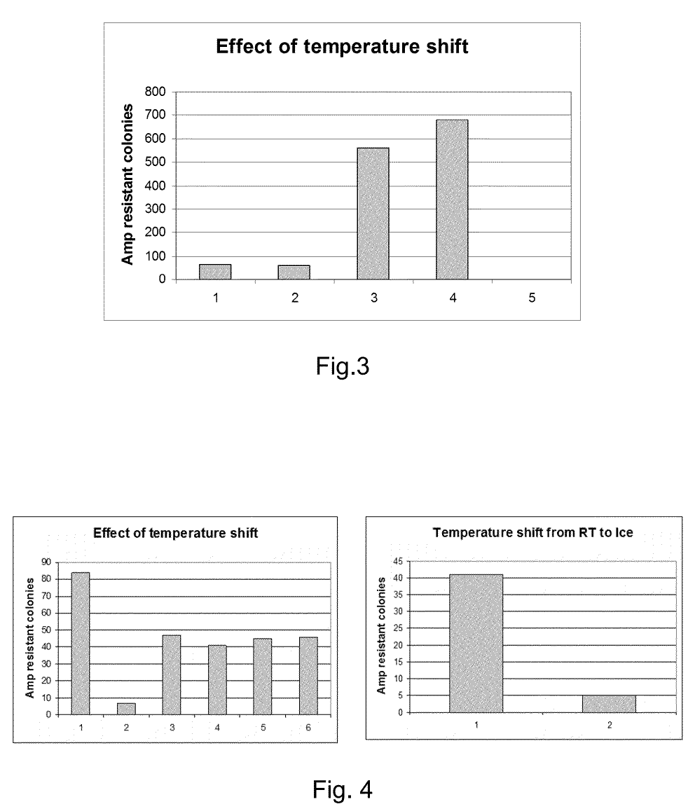

Electrocompetent cells were prepared and electroporated at different temperatures. GB2005 cells were transformed by about 0.1 μg of pGB-amp-Ptet-plu1880 (27.8 kb) and plated on Amp plates after 10⁴ times dilution. The result in FIG. 3 shows that transformation efficiency was dramatically increased when competent cells were shifted from ice-cold to room temperature (RT). GB2005 were grown at 37° C. for 3 hrs, reaching an OD600 at 0.6. Washing and electroporation were carried out using protocols in Example 9 except for the following:

TABLE 2

| | Experimental setup |
|---|---|
| 1 | wash the isolated cells two times by ice-cold water, use ice-cold |
| | cuvettes for electroporation immediately |
| 2 | wash the isolated cells two times by ice-cold water, leave cells |
| | on ice for 15 min before electroporation, use ice-cold cuvettes |
| | for electroporation |
| 3 | wash the isolated cells two times by ice-cold water, leave cells at |
| | room temperature for 15 min before electroporation, use room |
| | temperature cuvettes for electroporation |
| 4 | wash the isolated cells two times by room temperature water, |
| | leave cells at room temperature for 15 min before electroporation, |
| | use room temperature cuvettes for electroporation |
| 5 | wash the isolated cells two times by room temperature water, leave |
| | cells at room temperature for 15 min before electroporation, use |
| | room temperature cuvettes for electroporation, but use water (no |
| | plasmid DNA) for electroporation. |

Result for each experiment above is shown in FIG. 3.

### Example 11

### Effect of Temperature Shift

Electrocompetent cells were prepared and electroporated at different temperatures. GB2005 cells were transformed by about 0.1 μg of pGB-amp-Ptet-plu1880 (27.8 kb) and plated on Amp plates after 10⁵ times dilution. Preparation were carried out using protocols in Example 9 except for the following:

TABLE 3

| | Experimental setup |
|---|---|
| 1 | cells prepared at RT and electroporated at RT |
| 2 | cells prepared on ice and immediately electroporated at |
| | RT |
| 3 | cells prepared on ice first then left at RT for 2.5 min before |
| | electroporation. Electroporation at RT |
| 4 | cells prepared on ice first then left at RT for 4 min before |
| | electroporation. Electroporation at RT |
| 5 | cells prepared on ice first then left at RT for 10 min before |
| | electroporation. Electroporation at RT |
| 6 | cells prepared on ice first then left at RT for 15 min before |
| | electroporation. Electroporation at RT |

Result for each experiment above is shown in FIG. 4 left.

TABLE 4

| | Experimental setup |
|---|---|
| 1 | cells prepared at RT and electroporated at RT |
| 2 | cells prepared at RT, placed on ice for 5 min before electroporation, |
| | and electroporated at ice-cold condition. |

Result for each experiment above is shown in FIG. 4 right.

It was surprisingly found that ice-cold prepared competent cells have better transformation efficiency when the cells were shifted from ice-cold to RT (FIG. 4 left). The cells may be kept at room temperature at least up to 15 minutes without much efficiency loss (FIG. 4 left, column 3, 4, 5 and 6). On the other hand, when RT prepared competent cells were shifted from RT to ice-cold, the transformation efficiency dropped down 8 times (FIG. 4 right). The data clearly suggest that cold-prepared or cold-stored electrocompetent cells have less transformation efficiency than the electrocompetent cells at RT.

### Example 12

Storage under room temperature before electroporation GB2005 strain was cultured at 37° C. till OD600 to about 0.5. The cells were pelleted and washed by water which was kept at room temperatures for some time before electroporation. GB2005 cells transformed by about 0.1 μg of pGB-Ptet-plu1880 (27.8 kb) and plated on Amp plates (dilution 2.5×10⁵). Preparation were carried out using protocols in Example 9 except for the following:

TABLE 5

| | Experimental setup |
|---|---|
| 1 | cells electroporated immediately under room temperature |
| 2 | cells electroporated after 1 hour storage at room temperature |
| 3 | cells electroporated after 4 hour storage at room temperature |
| 4 | cells electroporated after 24 hour storage at room temperature |

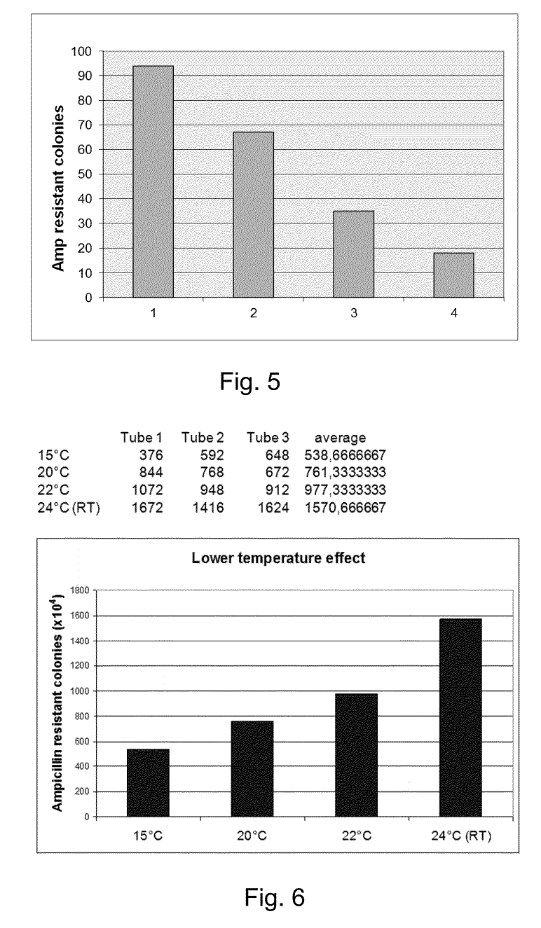

Result for each experiment above is shown in FIG. 5. As shown, electrocompetent cells prepared at room temperature can be kept at room temperature for longer period of time compared to cells prepared with conventional method (FIG. 4 left and Example 12). This shows that by preparing the cells under room temperature can prolong storage time.

### Example 13

### Effect of Temperatures Lower than 24° C.

GB2005 strain was cultured at 37° C. till OD600 to about 0.5. The cells were pelleted and washed by water which was kept at different temperatures (15° C., 20° C., 22° C. and 24° C.). The centrifuges and cuvettes were also cooled at these temperatures. GB2005 cells transformed by about 0.1 ug of pGB-Ptet-plu1880 and plated on Amp plates (dilution 5×10⁴). After electroporation with pGB-plu1880 plasmid (0.1 μg), 1 ml LB was added into the cuvette to recover the transformed cells for 1 hour at 37° C. 0.1 μl of cells were plated on ampicillin plates. Three independent tubes of competent cells were used for each temperature.

TABLE 6

| | Experimental setup |
|---|---|
| 1 | cells washed, centrifuged and electroporated at 15° C. |
| 2 | cells washed, centrifuged and electroporated at 20° C. |
| 3 | cells washed, centrifuged and electroporated at 22° C. |
| 4 | cells washed, centrifuged and electroporated at 24° C. |

Result for each experiment above is shown in FIG. 6. The figure shows that 24° C. is the best condition for preparation of the competent cells, and the transformation efficiency decreases as the temperature becomes lower.

### Example 14

### Effect of Temperatures (15-37° C. Compared to Conventional Method)

This example shows the effect of different temperatures on electrocompetent cells.

GB2005 strain was cultured at 37° C. till OD600 to about 0.5. The cells were pelleted and washed by water which was kept at different temperatures (2, 15, 20, 22, 24, 26, 28, 30, 32, 34 or 37° C.). The centrifuges and cuvettes were also kept at these temperatures. After electroporation with about pGB-plu1880 plasmid, 1 ml LB was added into the cuvette to recover the transformed cells. After cultured for 1 hour at 37° C., 0.004 μl of cells (diluted by LB) were plated on ampicillin plates. Three independent tubes of competent cells were used for each temperature. Numbers of colonies on the plates were averaged.

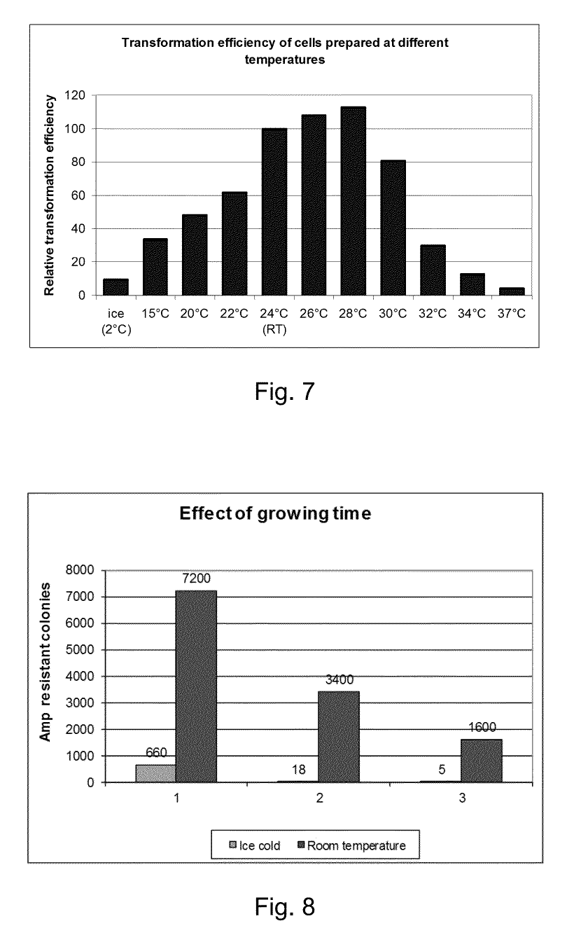

FIG. 7 shows the results in relative transformation efficiency using the transformants at 24° C. (room temperature) as standard (100%). Transformants from different temperatures were divided by standard to give the relative transformation efficiency. FIG. 7 shows that best results were obtained by preparing competent cells between 24° C.-28° C. This confirms that preparation of electrocompetent cells can be made as simple as possible.

### Example 15

### Effect of Growing Time

GB2005 cells grown to different phases were used to prepare electrocompetent cells using conventional method and present method. 35 ul overnight cultured GB2005 cells were diluted into 1.4 ml LB medium and cultured at 37° C. until different density is reached and then transformed by about 0.1 μg of pGB-Ptet-plu1880 and plated on Amp plates (dilution 10⁴).

The result is shown in FIG. 8 (1—cells growing for 2.5 hours, OD600=0.4; 2—cells growing for 4 hours, OD600 about 1.2; 3—cells growing for 6 hours, OD600 more than 1.8). For cells prepared using conventional ice cold method, the best transformation efficiency is observed for cells taken from the log phase (FIG. 8, column 1, OD600=0.4). The transformation efficiency is completely lost when electrocompetent cells from 4 hours (OD600 about 1.2) and 6 hours (OD600 more than 1.8) (only 18 and 5 colonies respectively). For cells prepared using the present method, best transformation efficiency was also observed for cells taken from the log phase. For cells taken after in the stationary phase (OD>1.2), transformation efficiency is still much better than prepared ice-cold.

### Example 16

### Omission of Recovery Step

GB2005 harvested at OD600=0.5 were used to prepare electrocompetent cells using conventional and present method). Cells were then transformed by about 0.1 μg of pGB-Ptet-plu1880 and plated on Amp plates (dilution 3.3×10⁴) directly or after the recovery step (incubation in1 ml LB medium at 37° C. for 1 hr).

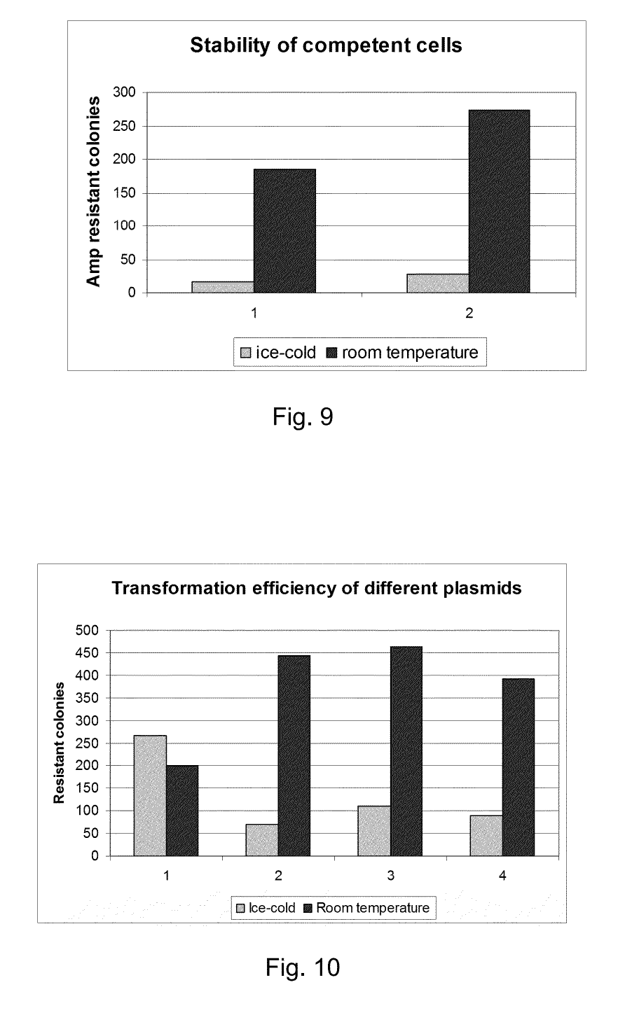

The result is shown in FIG. 9 (#1. without recovery step and #2. with recovery step). It was surprisingly found that for quick and simple plasmid transformation, it is not necessary to perform the recovery step when the electrocompetent cells were prepared at room temperature, and the result is still better than cells prepared by the conventional method with recovery step. This example illustrates that the recovery step may be omitted.

### Example 17

### Preparation from E. coli Strains and Transformation

E. coli strains XL-1 blue, DH5α, MM294, JM103 and GB2005 were used for DNA cloning and transformation. Cells were prepared according to the present method (RT method) and conventional method (cold method) Cells were transformed by about 0.1 μg of pGB-Ptet-plu1880 and plated on Amp plates (dilution 5×10³).The protocols as described in Example 9 were used.

Table 7 shows the number of colonies appeared on the plates. All strains show higher competence when prepared at room temperature.

TABLE 7

| Strains | XL-1 blue | DH5α | MM294 | JM103 | GB2005 |
|---|---|---|---|---|---|
| Cold method | 204 | 2 | 1 | 0 | 1500 |
| RT method | 1900 | 87 | 56 | 13 | 4300 |

### Example 18

### Preparation of GB2005 and Transformation with Different Plasmids

### Example 18.1

### Transformation with Smaller Plasmids

GB2005 cells were prepared by the present RT method or cold method and subsequently transformed with different plasmids, and plated on Cm or Amp plates. The protocols in Example 9 were used but with different plasmids:

TABLE 8

| | Experimental setup |
|---|---|
| 1 | Transformation with p15A-cm (1.9 kb) |
| 2 | Transformation with p15A-amp (2.1 kb) |
| 3 | Transformation with pcDNA3 (5.0 kb) |
| 4 | Transformation with pcDNA6 (5.1 kb) |

The plasmid p15A-cm was derived from pACYC184. p15A-amp was derived from pACYC177. pcDNA3 and pcDNA6 were obtained from Invitrogen. Result for each experiment above is shown in FIG. 10.

### Example 18.2

### Transformation with Larger Plasmids

GB2005 cells were transformed by different plasmids with large inserts including BACs and plated on Cm or Amp plates (dilution 2.5×10⁵). pcDNA vectors are pUC origin based plasmids and p15A vectors are p15A origin based plasmids.

TABLE 9

| | Experimental setup |
|---|---|
| 1 | Transformation with pGB-amp-Ptet-plu1880 plated on Amp plates |
| | (pUC origin, 27.8 kb) (dilution: 2.5 × 10⁵) |
| 2 | Transformation with p15A-amp-cm-plu2670 plated on Cm plates |
| | (p15A origin, 54.7 kb) (dilution: 5 × 10⁴) |
| 3 | Transformation with p15A-amp-cm-plu2670 plated on Amp plates |
| | (p15A origin, 54.7 kb) (dilution: 5 × 10⁴) |
| 4 | Transformation with MLL BAC on Cm plates |
| | (BAC origin with Cm resistance, >120 kb) (dilution: 500x) |
| 5 | Transformation with Rhi BAC on Km plates |
| | (BAC origin with Km and Amp both resistance, 91.7 kb) |
| | (dilution: 500x) |
| 6 | Transformation with Rhi BAC on Amp plates |
| | (BAC origin with Km and Amp both resistance, 91.7 kb) |
| | (dilution: 500x) |

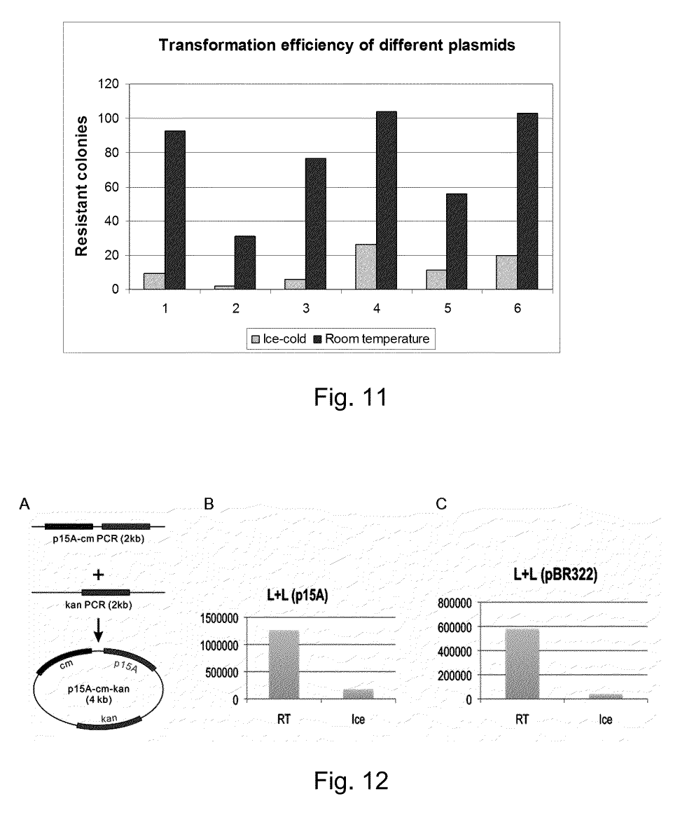

Result for each experiment above is shown in FIG. 11.

In all but one instance (p15A-cm) the cells have better transformation efficiency when prepared at room temperature. This suggests that transformation efficiency can be improved by preparation at RT.

### Example 19

### Electrocompetent Cells for Recombineering

### Example 19.1

### Linear to Linear Recombination

Recombineering is a DNA engineering technology via homologous recombination in vivo mediated by RecET or lambda Red Gam Beta Alpha (red gba). One of the limiting factors for recombineering is transformation efficiency because both molecules (donor and recipient) must meet each other in one cell by electroporation.

Linear to linear recombination (“LLHR”) is the method for cloning of PCR product or DNA fragment (diagrammed in FIG. 12A). A PCR product of linear vector (p15A ori-cm or pBR322 ori-cm) and a PCR product of km was built and used to test LLHR efficiency. GB05-dir (Fu et al. Enhanced direct DNA cloning by full length RecE and its application to secondary metabolites. Nature Biotechnology. 30(5):440-446 (2012)) carries recETgA operon in its chromosome and it is used for LLHR test. The protocols used were described in Example 9.1 and 9.2, however, with additional induction by adding 40 ml 10% L-arabinose after 2 hours culturing at 37° C. The tube is then additionally incubated for 45 min. Furthermore, after adding 1 ml of LB medium into the cuvette and transfer back into the Eppendorf tube, the tube is incubated at 37° C. for 70 mins with shaking.

TABLE 10

| | Experimental setup |
|---|---|
| L + L | 100 μg linear vector (p15A ori-cm) plus 100 ug km PCR |
| (p15A) | product were co-electroporated into the GB05-dir |
| | electrocompetent cells and plated on Km plates after 1 hour |
| | recovery |
| L + L | 100 μg linear vector (pBR322 ori-cm) plus 100 ug km PCR |
| (pBR322) | product were co-electroporated into the GB05-dir |
| | electrocompetent cells and plated on Km plates after 1 hour |
| | recovery |

Results are shown in FIGS. 12B and 12C for L+L (p15A) and L+L (pBR322), respectively. The Y axle represents numbers of colonies.

As shown, LLHR efficiency is 4 to 10 times more when the cells were prepared at RT. The increase is observed for both plasmid origins (p15A and pBR322). Example 19.1 shows electrocompetent cells prepared at RT exhibit improved LLHR efficiency.

### Example 19.2

### Effect of Length of Homology Arms in LLHR

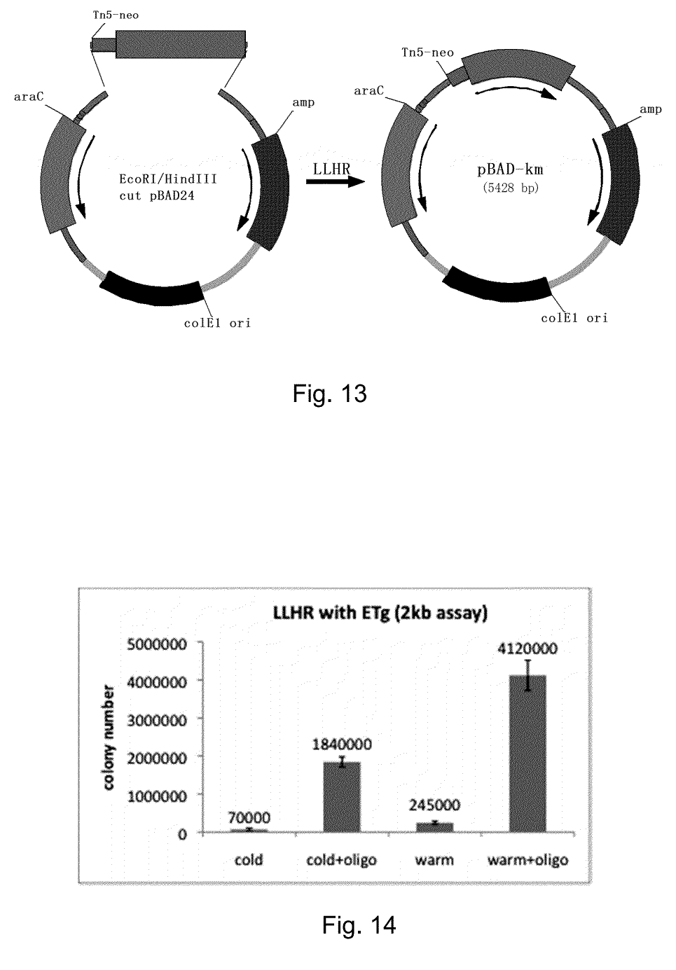

Previously 21 bp was taught as the minimum homology sequence for LLHR (Zhang et al., DNA cloning by homologous recombination in Escherichia coli. Nat. Biotechnol. 18, 1314-1317 (2000)). To test whether the minimal length could be even shortened, two plasmids were built: pBAD24 vector was digested with EcoR I+Hind III as linear recipient (FIG. 13). The homology sequences are exactly exposed at the ends. Tn5-neo PCR product flanked with short homology arms to the ends of digested pBAD24 vector is used as linear donor fragment. The short homology sequences at both ends of linear vector were flanked at the PCR product. Seven PCR products with different homology arms (HA) were used for testing the LLHR efficiency (Table 11. Numbers in the table represent the colonies on Amp+Km plates after LLHR). The homology arms can be as short as 8 bp for LLHR to occur when cells were prepared at RT (Table 11). When ice-cold cells used, the minimum homology arms are 12 bp. These data indicate that it is possible to use the present invention to provide a kit for PCR product or small DNA fragment cloning by using homology arms as short as 8 bp.

TABLE 11

| Length of HA | 6 bp | 8 bp | 10 bp | 12 bp | 14 bp | 18 bp | 22 bp |
|---|---|---|---|---|---|---|---|
| RT method | 0 | 20 | 78 | 278 | 909 | 3090 | 9380 |
| Cold method | 0 | 0 | 0 | 15 | 62 | 239 | 780 |

### Example 19.3

### LLHR with Carrier Oligo Using Cells Prepared Cold or Warm

LLHR efficiency can be drastically improved by adding additional single-stranded oligonucleotides (carrier oligo) with non-homology to linear molecules (linear vector and PCR product), as described in WO2011/154927.

In this example LLHR was performed in GB2005 with pSC101-BAD-ETg (tet) (Fu et al Nature Biotechnology 30(5):440-446 (2012)). Carrier oligo sequence is 5′ GGACGTTCCATTAGATCTGACTGCACCGCGTGACTAACGT 3′ (40 nt) and 100 pmol was used per electroporation.

Results shows that electrocompetent cells prepared by the present method (warm) is better than the conventional cold method (cold) in LLHR, with or without carrier oligo (FIG. 14).

This example demonstrates that carrier oligos increase LLHR efficiency using the electrocompetent cells prepared at RT. The highest LLHR efficiency is from electrocompetent cells prepared by RT protocol plus carrier oligo. Kit provided by the present invention may advantageously include carrier oligos.

### Example 19.4

### Linear to Circular Recombination

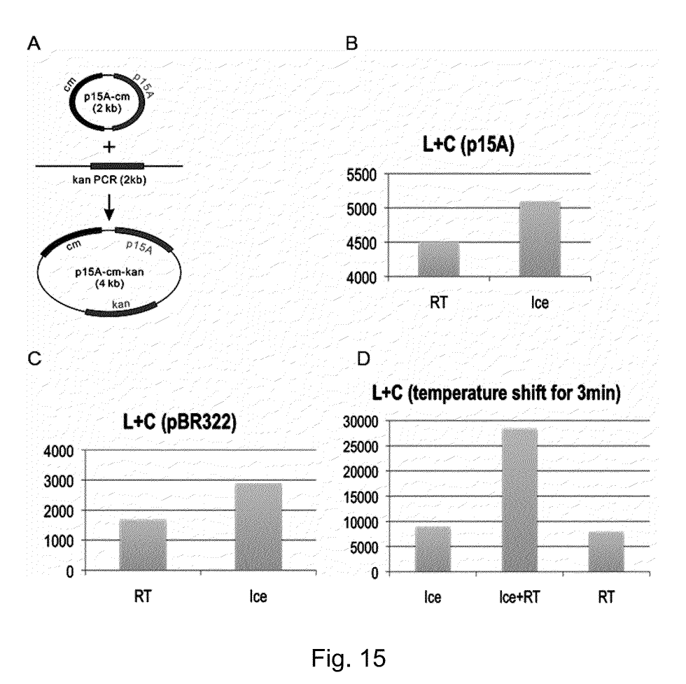

Linear to circular recombination (LCHR, FIG. 15A) is the method for plasmid, BAC or chromosome engineering by using recombineering. GB05-red derived from GB2005 carries red operon plus recA (gbaA) in its chromosome. This cell is used for testing LCHR recombineering efficiency. GB05-red is described in WO2011/154927 and Fu et al 2012.

The protocols used corresponds to the description in Example 9.1 and 9.2, however, with additional induction by adding 40 ml 10% L-arabinose after 2 hours culturing at 37° C. The tube is then additionally incubated for 45 min. Furthermore, after adding 1 ml of LB medium into the cuvette and transfer back into the Eppendorf tube, the tube is incubated at 37° C. for 70 mins with shaking. Further differences are described below.

FIG. 15 shows LCHR efficiency by using GB05-red electrocompetent cells. A, diagram of LCHR by using p15A ori-cm or pBR322 ori-cm circular vector plus km PCR product. B, 100 ug circular vector plus 100 ug km PCR product were co-electroporated into the GB05-red electrocompetent cells and plated on Km plates after 1 hour recovery. C, the same as B but circular vector is pBR322 ori-cm. D, pBR322 ori-cm is used as circular vector. The electrocompetent cells were prepared on ice first. After adding Km PCR product into the ice-cold electrocompetent cells, the cells+DNA mixture were shifted to RT for longer than 3 minutes before electroporation (middle column).

Electrocompetent cells prepared at RT shows lower LCHR efficiency (FIG. 15BC). But when the ice-cold electrocompetent cells plus DNA were placed at RT for longer than 3 minutes to make the mixture reach RT, the LCHR efficiency is increased 3.5 folds (FIG. 15D). This data indicates that red recombinases (Red alpha and beta) are not stable or decayed during preparation of electrocompetent cells at RT. But as shown in Example 19.3 above, GB-red electrocompetent cells can be prepared using cold method and electroporated at room temperature for the purpose of LCHR.

### Example 20

### Storage and Delivery of Electrocompetent Cells

Electrocompetent cells were prepared from GB05-dir and used for LLHR and transformation. The protocol for electrocompetent cells preparation is the same as described in Example 9.1 and 9.2 with the difference that after washing two times by room temperature dH₂O or 10% glycerol, cells were pelleted once again and remaining dH₂O or 10% glycerol was removed by pipetting. Undried cells, dried cells (0, 1 and 3 days) were tested for transformation efficiency and LLHR efficiency. If dried, cell pellets were dried under the vacuum for 30 min and stored at 4° C. pGB-amp-plu1880 (about 28 kb) was used in transformation, and linearized pBAD24 plus km PCR product with 12 bp homology arms (as described in Example 19.2) were used in LLHR assay.

Dried cells were resuspended in 25 μl dH₂O (no glycerol) at room temperature and DNA was added into the cells for transformation. Cells and DNA were electroporated at 1300v by using 1 mm-gap electroporation cuvette and Eppendorf electroporator as described in Example 19.2.

Results are shown in Table 12-13.

TABLE 12
Transformation efficiency (colonies on plates with ampicillin (×10⁴))
using cells prepared in dH₂O or 10% glycerol

| | Cells | Dried | Dried | |
|---|---|---|---|---|
| | before dry | cells day 0 | cells day 1 | Dried cells day 3 |
| dH₂O | 640 | 0 | 0 | 0 |
| 10% glycerol | 468 | 196 | 212 | 188 |

TABLE 13
LLHR efficiency (colonies on plates with ampicillin and kanamycin) using
cells prepared in dH₂O or 10% glycerol

| | Cells | Dried | Dried | |
|---|---|---|---|---|
| | before dry | cells day 0 | cells day 1 | Dried cells day 3 |
| dH₂O | 420 | 0 | 0 | 0 |
| 10% glycerol | 360 | 298 | 272 | 284 |

The RT electrocompetent GB05-dir cells prepared in dH₂O lost the transformation and LLHR efficiency if dried (row dH₂O in Table 12 and 13). The cells prepared in 10% glycerol are preferred for transformation and LLHR (row glycerol in Table 12 and 13). Although dried cells lost half efficiency of transformation compared to the cells before dried (row glycerol in Table 12), the efficiency is good enough for transformation. About 20% of LLHR efficiency is lost after cells dried, but for short homology PCR cloning (12 bp), around 80% efficiency were still obtained per electroporation (row glycerol in Table 13).

Conventionally, electrocompetent E. coli cells were prepared on ice-cold and quickly frozen in liquid nitrogen and stored in −80° C. freezer. The electrocompetent cells are delivered in a dry-ice package. However, it was found the dried electrocompetent cells can be stored at 4° C. for longer than 3 days without significant loss of transformation efficiency. This data suggests that it is possible to deliver the cells on normal (wet) ice, which is easier and cheaper.

### Example 21

### Effect of Amount of Cells Used Per Transformation

GB2005 cells were rendered competent using the RT method. Different amount of pUC19 plasmids were added to the cell for transformation. The protocol used is described in Example 9.2.

TABLE 14

| | 10 ng | 1 ng | 100 pg | 10 pg | 1 pg | 0.1 pg |
|---|---|---|---|---|---|---|
| Colony number | 3.8 × 10⁷ | 1.6 × 10⁷ | 9 × 10⁵ | 5.3 × 10⁴ | 1.4 × 10⁴ | 5.6 × 10³ |
| Colony number per | 3.8 × 10⁹ | 1.6 × 10¹⁰ | 9 × 10⁹ | 5.3 × 10⁹ | 1.4 × 10⁶ | 5.6 × 10¹⁰ |
| microgram plasmid | | | | | | |
| pUC19 DNA | | | | | | |

Results show that the colony number of room temperature prepared electrocompetent competent cells can be up to 5.6×10¹⁰, which is at least 5 times better than commercial available electrocompetent cells.

### Example 22

### Transformation Protocol

A standard transformation protocol is provided for the purpose of the present invention for the measurement of absolute transformation efficiency.
1. Provide electrocompetent cells (generally between 10⁸ to 10¹⁰ cells).
2. Add 50 pg of pUC19 to the electrocompetent cells and place mixture into an RT electroporation cuvette (1 mm gap).
2. Electroporate the cells at 1300V using an Eppendorf electroporator.
3. Add 1 ml of LB medium into the cuvette and transfer back into the Eppendorf tube.
4. Incubate at 37° C. for 70 min with shaking.
5. Plate the cells on LB agar plates containing suitable antibiotic as well as LB agar plates without antibiotics.
6. Incubate the plates at 37° C. overnight.
7. Count the numbers of colony forming units on both plates.

Absolute transformation efficiency is calculated by dividing the total number of transformants (on antibiotic-containing LB) by the total number cells (on antibiotic-free LB (without selection pressure)).

### Example 23

### Comparison of Transformation Efficiency of Commercially Available Electrocompetent Cell and GB2005

Transformation efficiency of a commercially available electrocompetent cell XYZ (prepared cold by the provider) and GB2005 (prepared warm) were transformed by 50 pg pUC19. XYZ electrocompetent cell is known to have high transformation efficiency and is therefore used to compare to GB2005. XYZ cells were thawed on ice and 30 μl were used for each transformation. Electroporation and recovery is performed as described in example 22 except that the electroporation is carried out on ice. GB2005 cells were prepared electrocompetent cells under RT following example 9.2; electroporation and recovery is performed as described in example 22. Colony numbers on LB-Amp plates and LB were counted. The results (average of 2 samples) are shown in the table below.

TABLE 15

| | Commercially | |
|---|---|---|
| | available | |
| | electrocompetent | |
| | cell XYZ | GB2005 |
| Colony number on LB plates | 2.88 × 10¹⁰ | 6.40 × 10⁸ |
| Colony number on LB-amp plates | 9.60 × 10⁴ | 6.24 × 10⁵ |
| Absolute transformation efficiency | 3.68 × 10⁻⁶ | 9.75 × 10⁻⁴ |
| (colony/microgram pUC19/cell) | | |
| Transformation efficiency | 1.92 × 10⁹ | 1.25 × 10¹⁰ |
| (colony/microgram pUC19) | | |

Transformation efficiency: GB2005 prepared by warm protocol is 6.5 times better than the commercially available cell XYZ.

Absolute Transformation efficiency: GB2005 prepared by warm protocol is 265 times better than the commercially available cell XYZ.

This example demonstrates that GB2005 electrocompetent cells provided by the present invention are far better in terms of transformation efficiency as well as absolute transformation efficiency.

### Example 24

### Transformation Efficiency of Commercially Available Electrocompetent Cell Under Room-Temperature

The commercially available electrocompetent cell XYZ (prepared cold by the provider) was transformed. XYZ cells were thawed on ice and 30 μl was used. 50 pg pUC19 was added to the cells and the mixture were kept under room temperature for 5 mins. Electroporation and recovery is performed under room temperature as described in example 22. The results (average of 2 samples) are shown in the table below.

TABLE 16

| | Commercially available |
|---|---|
| | electrocompetent cell XYZ |
| Colony number on LB plates | 3.36 × 10¹⁰ |
| Colony number on LB-amp plates | 1.88 × 10⁵ |
| Absolute transformation efficiency | 5.60 × 10⁻⁶ |
| (colony/microgram pUC19/cell) | |
| Transformation efficiency | 3.76 × 10⁹ |
| (colony/microgram pUC19) | |

This example demonstrates that even if cells were prepared cold, electroporation at RT alone is able to increase the transformation efficiency (comparison with Table 15).

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A method of preparing electro competent cells from Gram-negative bacteria comprising:
a. growing Gram-negative bacterial cells in a medium, and
b. isolating the cells and washing the cells with water or a non-ionic liquid,
wherein the washing in step b) takes place under about 20° C. to about 30° C.

2. The method of claim 1, wherein the washing in step b) takes place under about 24 to about 28° C.

3. The method of claim 2, wherein the washing in step b) takes place under about 24 to about 25° C.

4. The method of claim 1, wherein the cells are grown until an optical density at 600 nm of more than 0.4.

5. The method of claim 4, wherein the cells are grown until an optical density at 600 nm of more than 1.6.

6. The method according to claim 1, wherein the cells are washed with about 20° C. to about 30° C. water.

7. The method according to claim 1, wherein the cells are washed with about 20° C. to about 30° C. glycerol.

8. The method according to claim 1, wherein the washing in step b) comprises suspending the cells in water or the non-ionic liquid, pipetting the cells up and down, and isolating the cells from the non-ionic liquid.

9. The method of according to claim 8, wherein the cells are isolated from water or the non-ionic liquid by centrifugation.

10. The method of according to claim 9, wherein the centrifugation is carried out under about 20 to about 30° C.

11. The method according to claim 1, wherein the washing in step b) is carried out at least 2 times.

12. The method according to claim 1, wherein the washing is carried out in a tube having about 20° C. to 30° C.

13. An electrocompetent cell Gram-negative bacterial produced by the of claim 1, said electrocompetent cell having an absolute transformation efficiency of at least 7×10⁻⁶.

14. The electrocompetent cell according to claim 13, wherein the Gram-negative bacterial cell is Proteobacteria.

15. The electrocompetent cell according to claim 14, wherein the Gram-negative bacterial cell is E. coli.

16. The electrocompetent cell according to claim 15, wherein the Gram-negative bacterial cell is GB2012, GB2005, GB05-dir or GB05-red.

17. The electrocompetent cell according to claim 14, wherein the Gram-negative bacterial cell is Salmonella, Burkholderia, Pseudomonas, Agrobacterium, Photorhabdus, Xenorhabdus or Myxobacterium.

18. The electrocompetent cell according to claim 14, wherein the Gram-negative bacterial cell is XL-1 blue, DH5α, DH5G, MM294, DH10B, HS996, JM103 or Top10.

19-20. (canceled)

21. A kit comprising packaging material, an electrocompetent cell of claim 13, and a control plasmid, wherein the packaging material maintains the electrocompetent cell at a temperature of −20° C. to 10° C., such that the kit has a delivery temperature of higher than −20° C.

22. (canceled)

23. A method of transforming Gram-negative bacteria comprising:
a. growing Gram-negative bacteria in a medium,
b. isolating the bacteria and washing the bacteria with water or non-ionic liquid, thereby obtaining electrocompetent cells, and
c. introducing nucleic acid molecules into the electrocompetent cells by electroporation, wherein the washing in step b) takes place under about 20 to about 30° C.

24-50. (canceled)

51. The method according to claim 1, further comprising a step c. wherein the cells are dried.

52. The method according to claim 51, further comprising a step d. wherein the cells are stored at a temperature of from about −80° C. to about 30° C.


SEQUENCE LISTING (1 sequence)

SEQ ID NO: 1 - DNA, 40 bp, artificial
  carrier oligo
ggacgttccattagatctgactgcaccgcgtgactaacgt